(19) United States
(12) Patent Application Publication
(10) Pub. No.: US 2026/0272881 A1
(43) Pub. Date: Sep. 17, 2026

(54) QTX125 DRUG FORMULATIONS

(71) Applicant: Quimatryx, S.L., San Sebastián, Guipúzcoa (ES)

(72) Inventors: Yosu Ion Salazar Vara, San Sebastián (ES); Eneko Arevalo Aldaba, San Sebastián (ES); Tamara Iglesias Bello, San Sebastián (ES); Laureano Buela Simon, San Sebastián (ES)

(73) Assignee: Quimatryx, S.L., San Sebastián, Guipúzcoa (ES)

(21) Appl. No.: 18/850,984

(22) PCT Filed: Mar. 31, 2022
(86) PCT No.: PCT/EP2022/058733

§ 371 Date: Sep. 25, 2024

(30) Foreign Application Priority Data
Mar. 29, 2022 (CN) 202210325433.9

(51) Int. Cl. A61K 31/4025; A61K 9/00; A61K 9/08; A61K 9/19; A61K 47/40

(52) U.S. Cl. CPC A61K 31/4025; A61K 9/0019; A61K 9/08; A61K 9/19; A61K 47/40

(57) ABSTRACT

The present invention relates to an aqueous pharmaceutical formulation for injection comprising:
a compound of formula I,

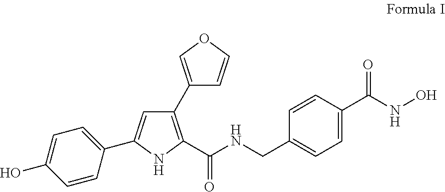 [SMILES: O=C(NO)c1ccc(CNC(=O)c2[nH]c(-c3ccc(O)cc3)cc2-c2ccoc2)cc1]

and a compound of formula II,

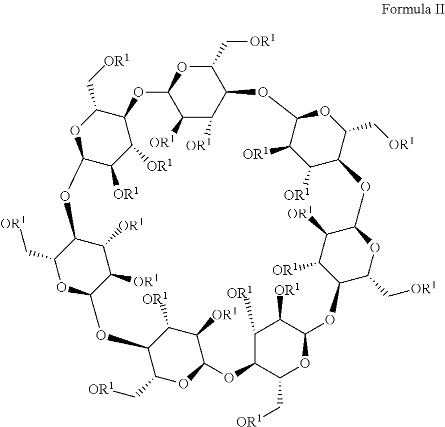

wherein each R¹ is independently selected from the group of: —H or

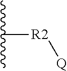

wherein R² is either absent or is a C₁₋₄alkyl;
Q is selected from the group of: —H, —SO₃⁻, —OH, —C(O)R³ or —C(OH)R³₂; and
R³ is independently selected from —H or is a C₁₋₄alkyl;
wherein the molar ratio of the compound of formula I to the compound of formula II is from 1:50-1:2; and
wherein the pH of the pharmaceutical formulation is between pH 7 and pH 8.

### FIELD OF THE INVENTION

This invention generally pertains to the field of pharmaceuticals and pharmacy and specifically to pharmaceutical formulations comprising a certain histone deacetylase inhibitor and a cyclodextrin or derivative thereof. The present invention also pertains to the use of such formulations, for example, in the treatment of cancer or autoimmune diseases.

### BACKGROUND

### Histone Deacetylase (HDAC)

Histone deacetylases (HDAC) constitute an interesting therapeutic target for the treatment of cancer (cf. P. A. Marks et al. Nature Rev. Cancer 2001, 1, 194; J. E. Bolden et al. Nature Rev. Drug Discov. 2006, 5, 769; P. Gallinari et al. Cell Res. 2007, 17, 195; K. B. Glaser Biochem. Pharmacol. 2007, 74, 659; L. Pan et al. Cell. Mol. Immunol. 2007, 4, 337; M. Haberland et al. Nature Rev. Genetics 2009, 10, 32; Y. Zhang et al. Curr. Med. Chem. 2008, 15, 2840; S. Ropero, M. Esteller Mol. Oncol. 2007, 1, 19) and other diseases such as those related to central nervous system, such as autoimmune diseases (cf. A. G. Kazantsev, L. M. Thompson Nature Rev. Drug Discov. 2006, 7, 854).

Several families of HDAC inhibitors (HDACis) have been designed, whose general structures can be found in different reviews (cf. A. Villar-Garea, M. Esteller Int. J. Cancer 2004, 112, 171; T. A. Miller et al. J. Med. Chem. 2003, 46, 5097; T. Suzuki, N. Miyata Curr. Med. Chem. 2005, 12, 2867; M. Paris et al. J. Med. Chem. 2008, 51, 1505). The general structure of these inhibitors consists of a cyclic structure, a spacer and a chelating group capable of binding to the Zn (II) cation of the active centre of the different HDAC isoforms that belong to the class I (HDAC1, HDAC2, HDAC3 and HDAC8), class II (HDAC4, HDAC5, HDAC6, HDAC7, HDAC9 and HDAC10) and class IV (HDAC11).

The mechanism of action of the HDAC inhibitors is explained by their antagonist properties against histone deacetylases involved in the regulation of processes related to apoptosis, cell growth, tumour progression, cancer metastasis, cell adhesion and others. These properties prevent the binding of HDACs to their natural ligands, which can be histones or cytoplasmic proteins such as tubulin, as well as their normal catalytic activation, namely the deacetylation of ε-N-acetyl lysine residues present in these proteins.

Despite having a similar inhibition mode, occasionally some selectivity in the inhibition of different HDAC isoforms has been observed (cf. J. C. Wong et al. J. Am. Chem. Soc. 2003, 125, 5586; G. Estiu et al. J. Med. Chem. 2008, 51, 2898). The mentioned selectivity is of therapeutic interest (cf. K. V. Butler, A. P. Kozikowski Curr. Pharm. Design 2008, 14, 505; T. C. Karagiannis, A. El-Osta Leukemia 2007, 21, 61).

### HDAC Inhibitors

One important class of HDAC inhibitors are trisubstituted pyrrolic derivatives connected with the chelating groups through aromatic and heteroaromatic groups, as described for example, in WO2011/039353. These compounds have been shown to be effective in the treatment of cancer (cf. WO2011/039353).

In addition, there compounds have been shown to be effective in the treatment of several autoimmune diseases. For example, these compounds have been shown to be effective in animal models of autoimmune hepatitis and autoimmune encephalomyelitis (cf. WO 2018/087082).

An especially promising compound is 3-(3-Furyl)-N-{4-[(hydroxyamino) carbonyl]benzyl}-5-(4-hydroxyphenyl)-1 Hpyrrole-2-carboxamide (referred to herein as QTX125).

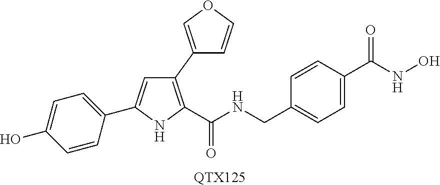

QTX125 is a highly selective and highly potent HDAC 6 inhibitor. It has shown high antitumoral efficacy in mantle cell lymphoma (cf. Perez-Salvia, M. et al haematologica 2018; 103:e540), lung cancer and pancreatic cancer xenograft murine models. QTX125 has also shown high efficacy in two different multiple sclerosis mice models (cf. WO 2018/087082).

However, hydroxamic acids such as QTX125 are known to have very low solubility in water (cf. Patre, S. et al. 2011 International Conference on Environment and BioScience IPCBEE vol. 21 (2011)) and in order to dissolve QTX125 in aqueous solution it is normally necessary to employ high pHs. QTX125 also demonstrates physical and chemical instability in solution.

For injectable formulations of QTX125 it is desirable to have a solubility of 7.5 mg/mL or more in a formulation at a physiological pH (i.e. from pH 7 to pH 8). This is particularly important from the point of view of avoiding pain at the injection sight, as compositions with particularly high or particularly low pHs tend to be more painful. Without being bound by any theory it is believed that formulations for infusion administration should be close to physiological pH range (pH 7-8) and for bolus administration should be from pH 4-8 for buffered solution and from pH 3-9 for unbuffered solution. Injectable formulations should also be stable over extended periods of time between manufacture and administration and have low toxicity.

Consequently, there remains a need in the art to provide pharmaceutical formulations comprising high concentrations of dissolved QTX125 at physiological pH which are stable and have low toxicity.

A number of patents and publications are cited herein in order to more fully describe and disclose the invention and the state of the art to which the invention pertains. Full citations for these references are provided herein. Each of these references is incorporated herein by reference in its entirety into the present disclosure.

### SUMMARY OF THE INVENTION

The present inventors have developed a pharmaceutical formulation, which helps to address the practical problems outlined above.

Accordingly, in a first aspect the present invention provides a pharmaceutical formulation for injection comprising:
a compound of formula I,

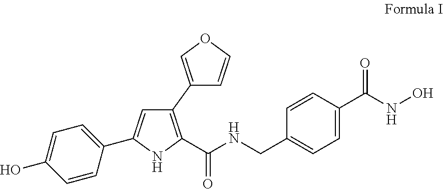 [SMILES: O=C(NO)c1ccc(CNC(=O)c2[nH]c(-c3ccc(O)cc3)cc2-c2ccoc2)cc1]

and a compound of formula II,

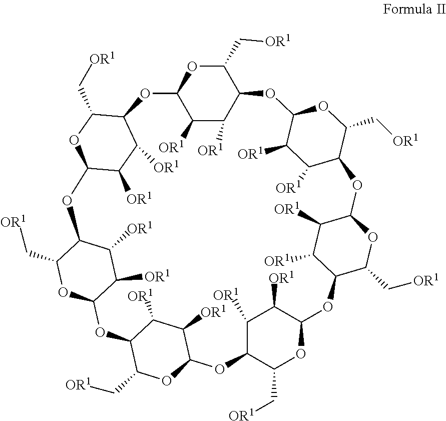

wherein each R¹ is independently selected from the group of: —H or

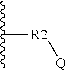

wherein R² is either absent or is a C₁₋₄alkyl;
Q is selected from the group of: —H, —SO₃⁻, —OH, —C(O)R³ or —C(OH)R³₂; and
R³ is independently selected from —H or is a C₁₋₄alkyl;
wherein the molar ratio of the compound of formula I to the compound of formula II is from 1:50-1:2; and
wherein the pH of the pharmaceutical formulation is between pH 7 and pH 8.

Preferably, the present invention relates to an aqueous pharmaceutical formulation for injection comprising a compound of formula I

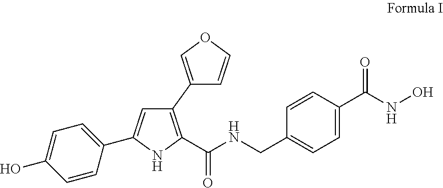 [SMILES: O=C(NO)c1ccc(CNC(=O)c2[nH]c(-c3ccc(O)cc3)cc2-c2ccoc2)cc1]

and sulfobutyl ether β cyclodextrin (SBβCD),
wherein the molar ratio of the compound of formula I to the sulfobutyl ether β cyclodextrin is from 1:50-1:2; and wherein the pH of the pharmaceutical formulation is between pH 7 and pH 8.

In a second aspect, the present invention relates to an aqueous pharmaceutical formulation for injection comprising:
a compound of formula I,

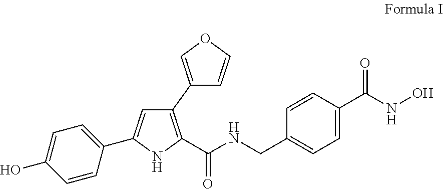 [SMILES: O=C(NO)c1ccc(CNC(=O)c2[nH]c(-c3ccc(O)cc3)cc2-c2ccoc2)cc1]

and compound of formula II,

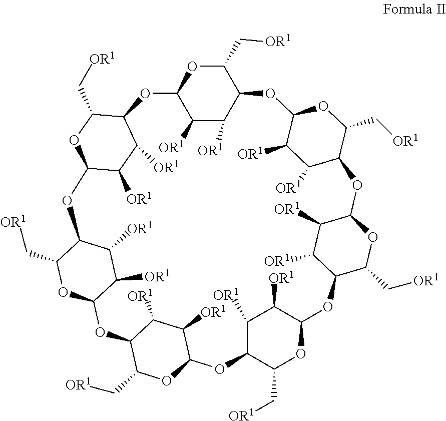

wherein each R¹ is independently selected from the group of: —H or

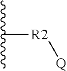

wherein R² is absent or is a C₁₋₄alkyl;
Q is selected from the group of: —H, —SO₃⁻, —OH, —C(O)R³ or —C(OH)R³₂ and
R³ is independently selected from —H or is a C₁₋₄alkyl
wherein the pharmaceutical formulation is obtainable by a method comprising:
   Step 1: preparing a mixture comprising water, a compound of formula I and a compound of formula II, wherein the molar ratio of the compound of formula I to the compound of formula II is from 1:50-1:2; and
   Step 2: adding an acid to lower the pH of the mixture to a value between pH 7 and pH 8.

In a further aspect, the present invention relates to a dry pharmaceutical formulation obtainable by drying an aqueous pharmaceutical formulation according to the present invention, preferably obtainable by lyophilising an aqueous pharmaceutical formulation according to the present invention.

In a further aspect, the present invention relates to a kit comprising the said dry pharmaceutical formulation and a pharmaceutically acceptable grade of water, buffer solution or saline solution for use in reconstituting the dosage form.

In a further aspect, the present invention relates to an aqueous pharmaceutical formulation or a dry pharmaceutical formulation as described herein, for use in the manufacture of a medicament.

In a further aspect, the present invention relates to an aqueous pharmaceutical formulation or a dry pharmaceutical formulation as descried herein for use in the manufacture of a medicament for the treatment of cancer or an autoimmune disease.

In a further aspect, the present invention relates to an aqueous pharmaceutical formulation or a liquid pharmaceutical formulation obtainable by reconstituting the dry pharmaceutical formulation as descried herein for use as a medicament.

In a further aspect, the present invention relates to an aqueous pharmaceutical formulation or a liquid pharmaceutical formulation obtainable by reconstituting the formulation as descried herein for use in the treatment of cancer or an autoimmune disease.

In a further aspect, the present invention relates to a method of treatment comprising administering an aqueous pharmaceutical formulation or a liquid pharmaceutical formulation obtainable by reconstituting the dry pharmaceutical formulation as descried herein to a patient.

In a further aspect, the present invention relates to a method of treatment of cancer or an autoimmune disease comprising administering an aqueous pharmaceutical formulation or a liquid pharmaceutical formulation obtainable by reconstituting the dry pharmaceutical formulation as descried herein to a patient.

As will be appreciated by one of skill in the art, features and preferred embodiments of one aspect of the invention will also pertain to other aspects of the invention.

### DETAILED DESCRIPTION

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “about” preceding a stated value indicates that the value may have an uncertainty of ±20%, preferably ±10%, ±5%, ±2%, ±1% of the stated value.

The term “C₁-C₄ alkyl” refers to a linear or branched hydrocarbon chain consisting of carbon and hydrogen atoms, containing no unsaturation, having from 1 to 4 carbon atoms, preferably between 1 and 3 (“C₁-C₃ alkyl”), and which is attached to the rest of the molecule through a single bond, including for example and in a non limiting sense, methyl, ethyl, n-propyl, i-propyl, n-butyl, t-butyl etc.

The term “room temperature” refers to the ambient temperature of a typical laboratory, which is typically between 20° C. and 30° C., preferably around 25° C. at atmospheric pressure.

The term “dry” refers to a formulation which has been subjected to drying. Optionally, a dry formulation may refer to a solid material with a residual water content of less than 10%, preferably less than 8%, preferably less than 5%, preferably from about 0.1% to about 5%. The residual water content may be determined using a Karl Fischer Titration.

The terms “lyophilised” or “freeze-dried” refer to substances obtained and/or obtainable by lyophilising a liquid formulation, that is from a drying procedure in which the material to be dried is first frozen followed by the removal of the ice or frozen solvent by sublimation under vacuum.

The term “reconstituted” refers to contacting the dry (lyophilised) formulation with a pharmaceutically acceptable liquid e.g. a pharmaceutically acceptable grade of water (preferably sterile), a pharmaceutically acceptable buffer solution or a saline solution, such that the powder (or solid compound) is converted to either a suspension or a solution which can be administered to a patient by injection. The term “reconstituted dry pharmaceutical formulation” relates to a liquid pharmaceutical formulation obtainable by reconstituting the dry pharmaceutical formulation according to the present invention in a pharmaceutically acceptable liquid.

The term “injection” refers to any form of injection known to a skilled person in the art such as subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal. Injection may refer to an infusion process (e.g. sustained administration) as well as bolus (discreate) administration.

The term “pharmaceutically acceptable salts” refers to salts which, when administered to the recipient, can provide (directly or indirectly) a compound as described in the present document. “Pharmaceutically acceptable” preferably refers to compositions and molecular entities that are physiologically tolerable and do not usually produce an allergic reaction or a similar unfavourable reaction such as gastric disorders, dizziness and suchlike, when administered to a human or animal. Preferably, the term “pharmaceutically acceptable” means it is approved by a regulatory agency of a state or federal government or is included in the U.S. Pharmacopoeia or other generally recognized pharmacopoeia for use in animals, and more particularly in humans.

The term “treatment” or “treating” refers to administration of a compound or a pharmaceutical composition of the invention to improve or eliminate the disease or one or more symptoms associated with the disease. The term “prevention” or “prevent” includes reducing the risk of the disease appearing or developing.

If not indicated otherwise “%” refers to weight-%.

The present invention has a number of advantageous features, including those listed below

Firstly, the aqueous pharmaceutical formulations according to the present invention mean that the compound QTX125 can be dissolved at a concentration of 7.5 mg/mL or more in a formulation with a pH of between pH 7 and pH 8, which is suitable for injection.

Secondly, the aqueous pharmaceutical formulations according to the present invention are non-toxic in mammals at dosages of up to 200 mg/kg and hence are suitable for use in methods of treatment in mammals, such as methods of treating cancer.

Thirdly, the aqueous pharmaceutical formulations according to the present invention are stable for extended periods of time, for example up to 3 months at 5° C. The aqueous pharmaceutical formulation is also able to be lyophilised without leading to any loss in activity of the formulation on reconstitution.

Fourthly, the dry (lyophilised) pharmaceutical formulations according to the present invention demonstrate high stability over periods of at least 3 months at room temperature, which could eliminate the need for cold chain transport of these formulations. These formulations are also stable and give a pH value between pH 7 and pH 8 once reconstituted even after extended periods of storage.

### Compound of Formula I

One aspect of the present invention is focused on providing an aqueous pharmaceutical formulation with the compound of formula I (QTX125) dissolved at high concentrations.

The aqueous pharmaceutical formulation comprises:
a compound of formula I,

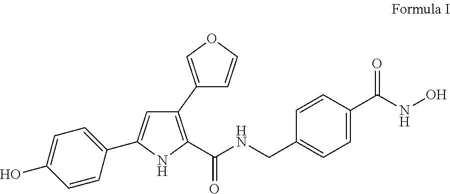 [SMILES: O=C(NO)c1ccc(CNC(=O)c2[nH]c(-c3ccc(O)cc3)cc2-c2ccoc2)cc1]

and a compound of formula II,

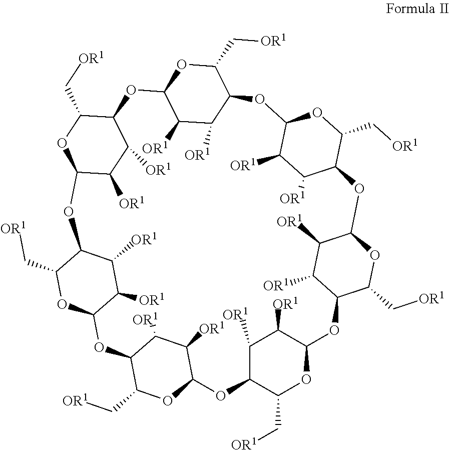

wherein each R¹ is independently selected from the group of: —H or

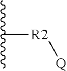

wherein R² is either absent or is a C₁₋₄alkyl;
Q is selected from the group of: —H, —SO₃⁻, —OH, —C(O)R³ or —C(OH)R³₂; and
R³ is independently selected from —H or is a C₁₋₄alkyl;
wherein the molar ratio of the compound of formula I to the compound of formula II is from 1:50-1:2; and
wherein the pH of the pharmaceutical formulation is between pH 7 and pH 8.

Preferably, the concentration of the compound of formula I dissolved in the aqueous pharmaceutical formulation is 7.5 mg/mL or more, more preferably 8 mg/mL or more, more preferably 8.5 mg/mL or more, more preferably 9 mg/mL or more and most preferably 9.5 mg/mL or more.

The concentration of QTX125 dissolved in the formulation may be determined using HPLC as described in the experimental section below.

The maximum concentration of QTX125 dissolved is limited only by the maximum solubility values of QTX125 which it is possible to achieve. Optionally, the maximum concentration of QTX125 in solution is 50 mg/mL, or 20 mg/mL, or 15 mg/mL.

The compound of formula I may be admixed into the aqueous pharmaceutical formulation in the form of a salt (preferably as pharmaceutically acceptable salt), as a solvate, as a free base, as a neutral compound or as a prodrug.

The preparation of salts can be accomplished by methods known in the art. For example, pharmaceutically acceptable salts may be synthesized from the original compound, which contains basic residues, by conventional chemical methods. Generally, such salts are prepared, for example, by reacting free base forms of the compound with the appropriate base or acid in water or in an organic solvent or in a mixture of both. In general, non-aqueous media like ether, ethyl acetate, ethanol, isopropanol or acetonitrile are preferred. Examples of acid addition salts include mineral acid addition salts such as, e.g. hydrochloride, hydrobromide, hydroiodide, sulfate, nitrate, phosphate salts and organic acid addition salts such as, for example, acetate, maleate, fumarate, citrate, oxalate, succinate, tartrate, malate, mandelate, methanesulfonate and p-toluenesulfonate salts. Examples of base addition salts include inorganic salts such as, for example, sodium, potassium, calcium, ammonium, magnesium, aluminum and lithium salts, and organic salts such as, for example, ethylenediamine, ethanolamine, N,N-dialkylethanolamine, triethanolamine, glucamine and basic salts of amino acids.

### Compound of Formula II

The aqueous pharmaceutical formulations according to the present invention further comprise a compound of formula II

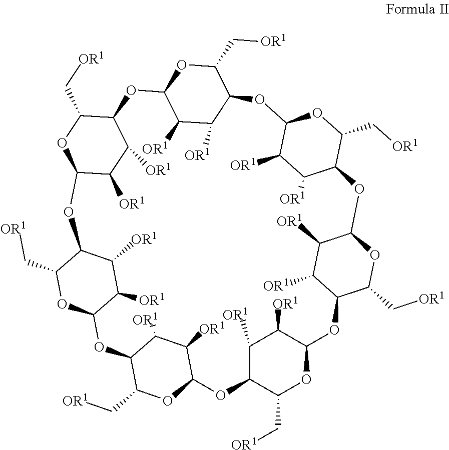

wherein each R¹ is independently selected from the group of: —H or

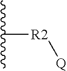

wherein R² is either absent or is a C₁₋₄alkyl;
Q is selected from the group of: —H, —SO₃⁻, —OH, —C(O)R³ or —C(OH)R³₂; and
R³ is independently selected from —H or is a C₁₋₄alkyl;

The compound of formula II is a β-cyclodextrin skeleton which may be substituted by a number of different functional groups (as set out above) on the hydroxy groups of the β-cyclodextrin. Cyclodextrins are cyclic oligosaccharides which possess a toroidal structure and harbour hydrophobic/lipophilic central cavities and hydrophilic outer surfaces. β-cyclodextrins are cyclodextrins consisting of 7 glucopyranose units.

A number of different types of β-cyclodextrins and their derivatives have been deemed safe for use as pharmaceutical excipients, for example 2-hydroxypropyl-β-cyclodextrin has been demonstrated to be well tolerated in a range of animal species including rats, mice and dogs (S. Gould et al., Food and Chemical Technology, 43, 1451-1459, 2005) and sulfobutyl ether β cyclodextrin (SBβCD) gave no observable effects in acute toxicity studies and no negative renal histopathology in mice (Rajewski, R. A., 1990. Development and evaluation of the usefulness and parenteral safety of modified cyclodextrins. Lawrence, KS, USA, Univ. Kansas, 251 pp)

Without being bound by any theory it is believed that the cyclodextrins interact with the QTX125 compounds to form a water-soluble complex, for example by incorporating the QTX125 molecule into their hydrophobic central cavity.

The compound of formula II is preferably selected from the group of β-cyclodextrin (naturally occurring β-cyclodextrin), (C₁₋₄alkyl)-β-cyclodextrin, sulfobutyl ethers of (C₁₋₄alkyl)-β-cyclodextrin, (hydroxy-C₁₋₄alkyl)-β-cyclodextrin, C₁₋₄alkyl-carboxylalkyl-β-cyclodextrin and C₁₋₄alkyl-(hydroxy-C₁₋₄alkyl)-β-cyclodextrin.

More preferably, the compound of formula II is selected from the group of β-cyclodextrin, (C₁₋₄alkyl)-β-cyclodextrin, (hydroxy-C₁₋₄alkyl)-β-cyclodextrin and sulfobutyl ethers of (C₁₋₄alkyl)-β-cyclodextrin.

More preferably, the compound of formula II is selected from the group of (hydroxy-C₁₋₄alkyl)-β-cyclodextrin and sulfobutyl ethers of (C₁₋₄alkyl)-β-cyclodextrin.

More preferably the compound of formula II is hydroxy propyl β cyclodextrin or sulfobutyl ether β cyclodextrin (SBβCD).

Most preferably the compound of formula II is sulfobutyl ether β-cyclodextrin (SBβCD).

Alternatively, the present invention may comprise two or more compounds according to formula II, for example the formulations according to the present invention may comprise both hydroxy propyl β cyclodextrin and sulfobutyl ether β cyclodextrin (SBβCD).

The compounds of formula II may be admixed as a free base or in a salt form, for example as a sodium salt or a potassium salt.

As will be appreciated by one of skill in the art, the average substitution pattern for cyclodextrins such as SBβCD may vary. The SBβCD may have on average 2-8 hydroxy groups substituted with a sulfobutyl ether moiety, preferably from 5-7 hydroxy groups substituted with a sulfobutyl ether moiety.

The average molecular weight of the compound of formula II varies depending on the degree of substitution. The average molecular weight of commercial preparations of sulfobutyl ether β-cyclodextrin (SBβCD) may vary from 1451-2242 g/mol.

### Molar Ratio of Components

In the aqueous pharmaceutical formulations according to the present invention, the molar ratio of the compound of formula I to the compound of formula II is from 1:50 to 1:2.

Preferably, the molar ratio of the compound of formula I to the compound of formula II is from 1:40 to 1:2, preferably from 1:30 to 1:2, preferably from 1:25 to 1:2, preferably from 1:20 to 1:2, preferably from 1:15 to 1:2, preferably from 1:10 to 1:2, preferably from 1:9 to 1:2, preferably from 1:8 to 1:2, preferably from 1:6 to 1:2, more preferably from 1:4.5 to 1:2. most preferably about 1:2.7.

In a certain aspect, the molar ratio of the compound of formula I to the compound of formula II is from 1:50 to 1:2.3.

Preferably, the molar ratio of the compound of formula I to the compound of formula II is from 1:40 to 1:2.3, preferably from 1:30 to 1:2.3, preferably from 1:25 to 1:2.3, preferably from 1:20 to 1:2.3, preferably from 1:15 to 1:2.3, preferably from 1:10 to 1:2.3, preferably from 1:9 to 1:2.3, preferably from 1:8 to 1:2.3, preferably from 1:6 to 1:2.3, more preferably from 1:4.5 to 1:2.3. most preferably about 1:2.7.

In a certain preferred aspect, the molar ratio of the compound of formula I to the compound of formula II is from 1:50 to 1:2.5.

Preferably, the molar ratio of the compound of formula I to the compound of formula II is from 1:40 to 1:2.5, preferably from 1:30 to 1:2.5, preferably from 1:25 to 1:2.5, preferably from 1:20 to 1:2.5, preferably from 1:15 to 1:2.5, preferably from 1:10 to 1:2.5, preferably from 1:9 to 1:2.5, preferably from 1:8 to 1:2.5, preferably between 1:6 to 1:2.5, more preferably from 1:4.5 to 1:2.5. most preferably about 1:2.7.

The concentration of the compound of formula II (e.g. sulfobutyl ether β-cyclodextrin) in solution may be determined using HPLC/MS based on comparison of the peak areas to a calibration curve of known compound.

### Other Additional Ingredients

Optionally, the aqueous pharmaceutical formulation additionally comprises one or more other pharmaceutically acceptable ingredients well known to those skilled in the art, including, but not limited to, pharmaceutically acceptable carriers, diluents, excipients, adjuvants, buffers, pH modifiers, preservatives, anti-oxidants, bacteriostats, stabilisers, suspending agents, solubilisers, surfactants (e.g., wetting agents), colouring agents, and isotonicizing solutes (i.e., which render the formulation isotonic with the blood, or other relevant bodily fluid, of the intended recipient). Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 18th edition, Mack Publishing Company, Easton, Pa., 1990; and Handbook of Pharmaceutical Excipients, 2nd edition, 1994.

Optionally, the aqueous pharmaceutical formulation according to the present invention further comprises a buffer (i.e. the formulation further comprises buffer salts dissolved therein). Optionally, the said buffer may be selected from the group of MES, Bis-Tris, ADA, ACES, PIPES, MOPSO, BES, MOPS, TES, HEPES, DIPSO, MOBS, TAPSO, Tris-HCl, HEPPSO, POPSO, TEA, EPPS, Tricine, Gly-Gly, Bicine, HEPBS, TAPS, AMPD, TABS, AMPSO, CHES, CAPSO, APS, CHAPS, CABS, Phosphate and histidine or a combination of the above.

Without being bound by any theory, it is believed that the use of a buffer may help to stabilise the composition at physiological pH.

The concentration of the buffer salt in the aqueous pharmaceutical formulation may range from 1 mM to 1 M, preferably 1 mM to 100 mM, preferably 5 mM to 50 mM, preferably 5 mM to 20 mM.

The aqueous pharmaceutical formulation may also comprise counter-ions and salts, such as sodium counter ions, chloride ions or NaCl dissolved is solution.

The aqueous pharmaceutical formulation may also comprise other active agents, for example, other therapeutic or prophylactic agents.

Optionally, the aqueous pharmaceutical formulation according to the present invention is substantially free of meglumine.

Optionally, the aqueous pharmaceutical formulation consists of
water
optionally salts, such as buffer salts or dissolved NaCl;
a compound of formula I,

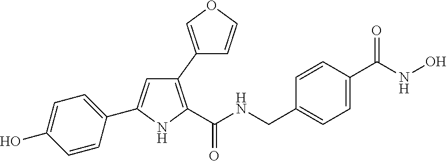 [SMILES: O=C(NO)c1ccc(CNC(=O)c2[nH]c(-c3ccc(O)cc3)cc2-c2ccoc2)cc1]

and
a compound of formula II,

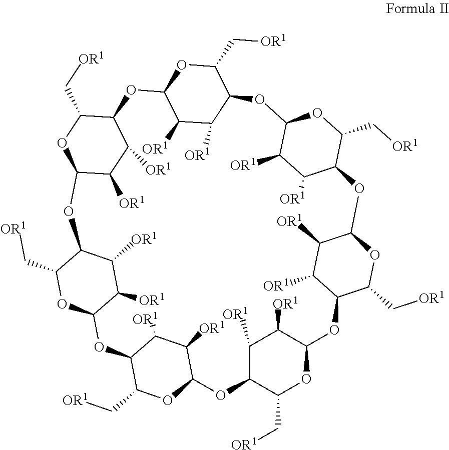

wherein each R¹ is independently selected from the group of: —H or

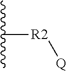

wherein R² is either absent or is a C₁₋₄alkyl;
Q is selected from the group of: —H, —SO₃⁻, —OH, —C(O)R³ or —C(OH)R³₂; and
R³ is independently selected from —H or is a C₁₋₄alkyl;
wherein the molar ratio of the compound of formula I to the compound of formula II is from 1:50-1:2; and
wherein the pH of the pharmaceutical formulation is between pH 7 and pH 8.

### Aqueous Pharmaceutical Compositions

Preferably, the aqueous pharmaceutical formulation of the present invention is obtainable by a method including:
Step 1: preparing a mixture comprising water, a compound of formula I and a compound of formula II, wherein the molar ratio of the compound of formula I to the compound of formula II is from 1:50-1:2;
Step 2: adding an acid to lower the pH of the mixture to a value between pH 7 and pH 8.

Preferably, step 1 is carried out at a pH of 9 or above. This pH may be set using any pharmaceutically acceptable base, such as sodium hydroxide.

Preferably, step 1 is carried out at a pH of 10 or above, preferably at a pH of 10.5 or above, preferably at a pH of 10.7 or above, preferably at a pH of 11 or above.

Any pharmaceutically acceptable acid may be used to lower the pH in step 2, such as 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p) and/or undecylenic acid.

Within the meaning of the present invention an aqueous pharmaceutical formulation may also refer to a reconstituted liquid pharmaceutical formulation obtainable by reconstituting the dry pharmaceutical formulation according to the present invention (as described below) in a pharmaceutically acceptable grade of water, buffer solution or salt.

### Dry Form

The present invention also relates to a dry pharmaceutical formulation obtainable by drying an aqueous pharmaceutical formulation according to the present invention.

Without wanting to be bound by any theory, it is believed that by drying the aqueous formulation according to the present invention it is possible to increase the stability of the compound (QTX125) and to ensure that the pH of the liquid pharmaceutical composition is between pH 7 and pH 8 when reconstituted.

The dry pharmaceutical composition according to the present invention comprises a compound of formula I and a compound of formula II in a molar ratio of from 1:50 to 1:2.

The drying step may be accomplished by any drying method known to a skilled person in the art, such as lyophilisation or spray drying. Preferably the drying step is carried out by lyophilising the aqueous pharmaceutical formulation according to the present invention.

Preferably, the dry pharmaceutical formulation is obtainable by a method including:
Step 1: preparing a mixture comprising water, a compound of formula I and a compound of formula II, wherein the molar ratio of the compound of formula I to the compound of formula II is from 1:50-1:2;
Step 2: adding an acid to lower the pH of the mixture to a value between pH 7 and pH 8;
Step 3: drying the solution

Preferably, step 1 is carried out at a pH of 9 or above, preferably at a pH of 10 or above, preferably at a pH of 10.5 or above, preferably at a pH of 10.7 or above, preferably at a pH of 11 or above.

Without wanting to be bound by any theory, it is believed that by dissolving the compound of formula I at a high pH the compound of formula I is incorporated into the cavity of the compound of formula II (the β-cyclodextrin). The compound of formula I remains incorporated in the cavity during steps 2 and 3 meaning that when the solution is reconstituted the compound of formula I is soluble in the aqueous solution at physiological pH.

The dry pharmaceutical formulation may be reconstituted into an aqueous pharmaceutical formulation according to the present invention by reconstituting the formulation in a pharmaceutically acceptable grade of water, a buffer solution or saline solution to give an aqueous pharmaceutical composition.

Alternatively, the dry pharmaceutical composition may be obtainable by a method including:
Step 1: preparing a mixture comprising water, a compound of formula I and a compound of formula II, wherein the molar ratio of the compound of formula I to the compound of formula II is from 1:50-1:2;
Step 2: drying the solution.

In this case, the reconstitution step is preferably carried out with an acidic solution to ensure that the solution obtained is at a pH suitable for injection.

### Preparation of Compositions

The present invention also relates to a method of manufacture of an aqueous pharmaceutical formulation according to the present invention, wherein the method comprises the steps of:
Step 1: preparing a mixture comprising water, a compound of formula I and a compound of formula II;
Step 2: adding an acid to lower the pH of the mixture to a value between pH 7 and pH 8.

Preferably step 1 is carried out at a pH of 9 or above, preferably at a pH of 10 or above, preferably at a pH of 10.5 or above, preferably at a pH of 10.7 or above, preferably at a pH of 11 or above.

Preferably, the concentration of the compound of formula II in step 1 is from 10 mg/mL to 2000 mg/mL, more preferably from 50 mg/mL to 1500 mg/mL, most preferably from 100 mg/mL to 1000 mg/mL

Preferably, step 1 comprises:
Step 1a: preparing a mixture comprising water and the compound of formula II;
Step 1b: adding a base to the mixture to ensure that it is at a pH of 9 or above, preferably at a pH of 10 or above; preferably at a pH of 11 or above
Step 1c: adding the compound of formula I.

Preferably, during step 1 the mixture is stirred using a stirring device until all of the compound of formula I has dissolved. The stirring device used is not particularly limited, suitable stirring devices may include a vortex mixer, a magnetic stirrer, a helix mixer or a paddle type stirrer.

Preferably, the mixture in step 1 is stirred for at least 40 minutes. Without wanting to be bound by any theory it is believed that this is the time necessary to ensure that the molecules of QTX125 have entered into the cavity in the cyclodextrin and break any intramolecular non-covalent interactions.

Optionally, when the method comprises steps 1a, 1b and 1c the mixture may be stirred for at least 20 minutes in step 1a, at least 15 minutes in step 1b and at least 40 minutes in step 1c.

Preferably, step 2 also involves diluting the mixture with a dilutant such as e.g. water.

Preferably, following step 2 the mixture is filtered, e.g. through a 0.45 μm or a 0.2 μm filter.

The method may also relate to a method of producing a dry pharmaceutical formulation comprising the steps of:
Step 1: preparing a mixture comprising water, a compound of formula I and a compound of formula II;
Step 2: adding an acid to lower the pH of the mixture to a value between pH 7 and pH 8;
Step 3: drying the mixture.

The preferred aspects relating to steps 1 and 2 described for the method of manufacture of an aqueous pharmaceutical formulation according to the present invention also apply to the method of producing a dry pharmaceutical formulation.

Preferably, the drying in step 3 is carried out by lyophilising the pharmaceutical formulation.

Optionally, the method may also involve step 4 storing the dry pharmaceutical formulation at room temperature for a period of at least three months.

The present invention also relates to an aqueous pharmaceutical formulation or a dry pharmaceutical formulation obtainable by the methods described above.

### Medical Use, Methods of Treatment

In a further aspect, the present invention relates to the aqueous pharmaceutical formulation or the dry pharmaceutical formulation according to the present invention for use in the manufacture of a medicament.

Preferably, the present invention relates to the aqueous pharmaceutical formulation or the dry pharmaceutical formulation according to the present invention for use in the manufacture of a medicament for the treatment of cancer.

Alternatively, the present invention relates to the aqueous pharmaceutical formulation or the dry pharmaceutical formulation according to the present invention for use in the manufacture of a medicament for the treatment of an autoimmune disease.

In a further aspect, the present invention relates to the aqueous pharmaceutical formulation or a liquid pharmaceutical formulation obtainable by reconstituting the dry pharmaceutical formulation according to the present invention for use as a medicament.

Preferably, the present invention relates to the aqueous pharmaceutical formulation or a liquid pharmaceutical formulation obtainable by reconstituting the dry pharmaceutical formulation according to the present invention for use in the treatment of cancer.

Alternatively, the present invention relates to the aqueous pharmaceutical formulation or a liquid pharmaceutical formulation obtainable by reconstituting the dry pharmaceutical formulation according to the present invention for use in the treatment of an autoimmune disease.

In a further aspect, the present invention relates to a method of treatment comprising administering an aqueous pharmaceutical formulation or a liquid pharmaceutical formulation obtainable by reconstituting the dry pharmaceutical formulation according to the present invention to a patient in need of such treatment.

Preferably, the present invention relates to a method of treating cancer comprising administering an aqueous pharmaceutical formulation or a liquid pharmaceutical formulation obtainable by reconstituting the dry pharmaceutical formulation according to the present invention to a patient in need of such treatment.

Alternatively, the present invention relates to a method of treating an autoimmune disease comprising administering an aqueous pharmaceutical formulation or a liquid pharmaceutical formulation obtainable by reconstituting the dry pharmaceutical formulation according to the present invention to a patient in need of such treatment.

Preferably, the cancer is selected from breast cancer, chronic myelogenous (or myeloid) leukemia (CML), colorectal cancer, lymphoma (such as non-Hodgkin lymphoma), fibrosarcoma, gastric cancer, glioblastoma, kidney cancer, liver cancer, lung cancer, melanoma, nasopharyngeal cancer, oral cancer, orthotopic multiple myeloma, osteosarcoma, ovarian cancer, pancreatic cancer, and prostate cancer.

Preferably, the autoimmune disease is selected from autoimmune hepatitis; an inflammatory demyelinating disease of the central nervous system; systemic lupus erythematosus; acute anterior uveitis; Sjogren's syndrome; rheumatoid arthritis; diabetes mellitus type 1; Graves' disease; and inflammatory bowel disease.

An inflammatory demyelinating disease of the central nervous system is a disease wherein myelin-supporting cells of the central nervous system, such as oligodendrocytes, and/or the myelin lamellae are destroyed. Demyelination leads to a disruption in neural signals between the brain and other parts of the body, ultimately resulting in a range of signs and symptoms, including physical, mental, and sometimes psychiatric problems.

Specific, non-limiting examples of inflammatory demyelinating diseases are multiple sclerosis (MS), including relapsing-onset MS, progressive-onset MS, optic-spinal MS; neuromyelitis optica; acute-disseminated encephalomyelitis; acute hemorrhagic leukoencephalitis; Balo concentric sclerosis; Schilder's disease; Marburg MS; tumefactive MS; solitary sclerosis; optic neuritis; transverse myelitis; Susac's syndrome; leukoaraiosis; myalgic encephalomyelitis; Guillain-Barre syndrome; progressive inflammatory neuropathy; leukodystrophy, including adrenoleukodystrophy and adrenomyeloneuropathy. Preferably, the autoimmune disease is multiple sclerosis or acute-disseminated encephalomyelitis. More particularly it is acute-disseminated encephalomyelitis, or more particularly and most preferably it is multiple sclerosis.

Preferably, the autoimmune disease is selected from autoimmune hepatitis and an inflammatory demyelinating disease of the central nervous system.

In a particularly preferred embodiment, the autoimmune disease is an inflammatory demyelinating disease of the central nervous system as described above.

In another particularly preferred embodiment, the autoimmune disease is autoimmune hepatitis.

The present inventors have found that QTX125, unlike other histone deacetylase inhibitors, advantageously show no evidence of genotoxicity, in particular of clastogenicity or aneugenicity. Similarly, it has unexpectedly been observed that QTX125 possess improved pharmacokinetic properties, in particular higher half-lives and distribution volumes, than other histone deacetylase inhibitors.

### Administration

Preferably, the aqueous pharmaceutical formulation according to the present invention (or liquid pharmaceutical formulation obtainable by reconstituting the dry pharmaceutical formulation according to the present invention) is administered via injection. The pharmaceutical formulation may be administered both via infusion (continuous) or bolus (discreate) administration.

The method of administration via injection may be, for example, subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal injection.

Preferably, the administration is by intravenous infusion or intravenous injection (bolus administration). More preferably, the administration is by intravenous infusion.

### Subject/Dosing

The subject for administration may be any animal. Preferably, the subject is a mammal, such as a rat, mouse, feline, canine, equine, porcine, ovine, bovine, primate or human. Preferably, the subject is a human patient.

In general, the effective amount of the compound of formula I to be administered will depended on a range of factors, such as the severity of the disorder being treated and the subject's weight. The active compounds will normally be administered one or more times a day for example 1, 2, 3, or 4 times daily, with typical total daily doses in the range from 0.01 up to 1,000 mg/kg/day.

Preferably, the compound of formula I is administered to human patients at a dosage of 0.5 to 50 mg/kg, preferably from 0.5 to 30 mg/kg, preferably from 1 to 20 mg/kg, more preferably from 5 to 10 mg/kg.

Preferably, the compound of formula I is administered to human patients at a dosage of from 25 mg to 4500 mg, preferably from 50 mg to 3000 mg, preferably from 250 mg to 1500 mg per day.

The compounds of the present invention can be used with at least one other drug to provide a combination therapy. This other drug or drugs may be part of the same composition, or may be provided as a separate composition and can be administered at the same time or at different times.

### Kits

Another aspect of the invention relates to a kit comprising a dry (preferably lyophilised) pharmaceutical formulation according to the present invention and a pharmaceutically acceptable grade of water, buffer solution or saline solution for use in reconstituting the dosage form. Preferably wherein the pharmaceutical composition is provided in a suitable container and/or with suitable packaging.

A kit may also include one or more delivery systems for delivering or administering the pharmaceutical composition provided therein e.g. a syringe and needle. The kit may also include directions for use (e.g. instructions for treating a subject).

Preferably, the kit also includes instructions for use, e.g. written instructions on how to administer the composition (e.g. the injection procedure). Most preferably, the kit includes written instruction on how to prepare a suitable pharmaceutical formulation from the dry pharmaceutical formulation (e.g. how to reconstituted the formulation) and how to subsequently administer the reconstituted pharmaceutical formulation.

As will be appreciated by one of skill in the art, features and preferred embodiments of one aspect of the invention will also pertain to other aspects of the invention.

### PREFERRED EMBODIMENTS

Particularly preferred embodiments include:

An aqueous pharmaceutical formulation for injection comprising:
a compound of formula I dissolved at a concentration of 9 mg/mL or more; and
sulfobutyl ether β-cyclodextrin
   wherein the molar ratio of the compound of formula I to sulfobutyl ether β-cyclodextrin is from 1:15 to 1:2.3.

Preferably, the ratio of the compound of formula I to sulfobutyl ether β-cyclodextrin is from 1:4.5 to 1:2.3.

Preferably, the aqueous pharmaceutical composition is obtainable by a method including:
Step 1: preparing a mixture comprising water, a compound of formula I and sulfobutyl ether β-cyclodextrin, wherein the molar ratio of the compound of formula I to sulfobutyl ether β-cyclodextrin is from 1:15-1:2.3 and optionally wherein the pH of the mixture is pH 9 or more;
Step 2: adding an acid to lower the pH of the mixture to a value between pH 7 and pH 8.

In a further preferred embodiment, the present invention relates to a dry pharmaceutical formulation, obtainable by lyophilising an aqueous pharmaceutical formulation comprising:
a compound of formula I dissolved at a concentration of 9 mg/mL or more; and
a compound of formula II
   wherein the molar ratio of the compound of formula I to the compound of formula II is from 1:15 to 1:2.3,
wherein the dry pharmaceutical formulation is obtainable by a method including:
   Step 1: preparing a mixture comprising water, a compound of formula I and a compound of formula II, wherein the molar ratio of the compound of formula I to the compound of formula II is from 1:50-1:2.3 and wherein the pH of the mixture is 9 or more, preferably 10.5 or more;
   Step 2: adding an acid to lower the pH of the mixture to a value between pH 7 and pH 8;
   Step 3: lyophilising the pharmaceutical formulation.

Preferably, the compound of formula II is sulfobutyl ether β-cyclodextrin (SBβCD).

Preferably, the pH of the mixture in step 1 is pH 10.7 or more.

In a further preferred embodiment, the present invention relates to a kit comprising the lyophilised pharmaceutical formulation described above and a pharmaceutically acceptable grade of water, buffer solution or saline solution for use in reconstituting the dosage form.

In a further preferred embodiment, the present invention relates to the use of a pharmaceutical formulation or a lyophilised formulation as described above, in the manufacture of a medicament.

Preferably, wherein the medicament is for the treatment of a cancer or an autoimmune disease.

More preferably, wherein the medicament is for the treatment of cancer.

### EXAMPLES

In order to better understand the nature of this invention, a number of illustrative examples will now be described.

The scope of the invention is not limited to the examples provided below. The examples merely demonstrate the effectiveness of the invention.

The experimental results from WO 2011/039353 are incorporated herein by reference.

### Experiment 1—Formulation Development

This example demonstrates how the solubility of various aqueous formulations comprising the compound of formula I

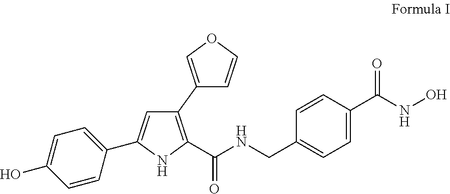 [SMILES: O=C(NO)c1ccc(CNC(=O)c2[nH]c(-c3ccc(O)cc3)cc2-c2ccoc2)cc1]

### (3-(3-Furyl)-N-{4-[(hydroxyamino) carbonyl]benzyl}-5-(4-hydroxyphenyl)-1H-pyrrole-2-carboxamide (QTX125))

vary depending on the formulation of the aqueous solution.

### Excipients

Parenteral grade excipients were used in the development of the formulations in example 1. Details of excipients are tabulated below in Table 1.

TABLE 1
Excipients details

| Ingredient | Manufacturer | Batch no./Lot no. |
|---|---|---|
| Sodium hydroxide | Avantor | 0000130791 |
| Hydrochloric acid | Merck KGaA | CG8C680514 |
| Glacial acetic acid | Avantor | 0000152649 |
| Meglumine | Spectrum | 3GD0046 |
| Sodium carbonate | Merck KGaA | DA6D660033 |
| L-arginine | Shanghai Kyowa | 17052625 |
| Phosphate buffered saline, | Sigma life sciences | SLB57363 |
| pH 7.20 | | |
| Dimethyl acetamide | Panreac | 869004 |
| Propylene glycol | Croda | 00012111510 |
| PVP K30/Kollidon 30LP | BASF | 12355816K0 |
| Hydroxy propyl β | Shandong binzhou | 20180502 |
| cyclodextrin | zhiyuan | |
| (Average molecular weight | biotechnology | |
| is 1541.5 g/mol) | | |
| Ethanol | Panreac | 827369 |
| L-arginine | Shanghai Kyowa | 17052625 |
| Meglumine | Spectrum | 3GD0046 |
| Sodium chloride | Merck KGaA | K48658924 705 |
| Sulfobutyl ether β | Cydex | NC-04A-160149 |
| cyclodextrin | Pharmaceuticals | |
| (Captisol) | | |
| (Average molecular weight | | |
| is 2161.7 g/mol) | | |
| Glycerin | Cargill | 06-03-30012018-013 |
| Glacial acetic acid | J T Baker (Avantor) | 0000152649 |
| Soybean oil | Croda | 0000993635 |
| Intralipid 20% v/v emulsion | Fresenius kabi | 8011007 |
| PEG 400 | BASF | 96402588QO |
| Water for injection | IH | NA |

0.2 μm filters were PDVF filters manufactured by Merck;
0.45 μm filters were nylon syringe filters manufactured by Merck

### Procedure for the Solubility Trials

The solubility trials in this application were carried out as follows:
The required amount of the excipients was weighed into a vial.
Diluent/cosolvent was added as per the composition of respective trial.
Contents were vortexed until solubilized.
The required quantity of QTX125 was weighed and added into the above mixture and vortexed for 10-15 min.
The volume of the formulation was made up using diluent.
The above solution was allowed to mix for 24 hours at room temperature using bottle rotating apparatus.

### Abbreviations

The following abbreviations are used in example 1.

| | |
|---|---|
| WFI | Water for injection |
| IH | In house |
| API | QTX125 (3-(3-Furyl)-N-{4-[(hydroxyamino) |
| | carbonyl]benzyl}-5-(4-hydroxyphenyl)-1 |
| | Hpyrrole-2-carboxamide) |
| IV | Intravenous |
| DMA | Dimethyl acetamide |
| PBS | Phosphate buffered saline |
| Qs | Quantity sufficient |
| HPβCD | Hydroxy propyl β cyclodextrin |
| SBβCD | Sulfobutyl ether β cyclodextrin |
| HCl | Hydrochloric acid |
| NaOH | Sodium hydroxide |

### Example 1.1—pH Modulation

In this example the effect of pH modulation was examined. The results are given in table 2.

TABLE 2
Formula composition of trials with pH modulation

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T1 | 50 mg API + 200 μL of | 10 | 12.34 | NA | Precipitation |
| | 2.0N sodium hydroxide + 4.80 | | | | observed - Not |
| | mL of Phosphate buffered | | | | analyzed |
| | saline (pH 7.20) | | | | |
| T2 | 50 mg API + 2.50 mL of | 5 | 10.70 | NA | Pale brown |
| | Phosphate buffered saline | | | | suspension - Not |
| | (pH 7.20) + 7.50 mL 0.5M | | | | analyzed |
| | Tromethamine | | | | |
| T3 | 1% API + 2% L- | 10 | 10.52 | 0.60 | White to light |
| | arginine + 97% WFI | | | | yellow colored |
| | | | | | turbid suspension |
| T4 | 1% API + 5% | 10 | 10.93 | 2.33 | White to light |
| | Meglumine + 94% WFI | | | | yellow colored |
| | | | | | turbid suspension |
| T5 | 50 mg API + 5.0 mL 0.1M | 10 | 10.25 | 0.07 | Whitish turbid |
| | tromethamine | | | | suspension |
| T6 | 50 mg API + 5.0 mL | 6.41 | 11.03 | 3.83 | Clear brown color |
| | WFI + 2.80 mL of 0.1N | | | | solution |
| | NaOH | | | | |
| T7 | 1% API + 200 μl of 1N | 10 | 11.79 | 6.02 | Pale brown color |
| | NaOH + Volume makeup | | | | clear solution |
| | with WFI | | | | |
| T8 | 1% API + 10% | 10 | 11.75 | 9.03 | Pale yellow color |
| | Meglumine + 10% of PVP | | | | clear solution |
| | K30 + 300 μl of 1N NaOH + | | | | |
| | Volume makeup with WFI | | | | |

From this data, it was observed that QTX125 forms a turbid suspension. The pH achieved was above 10 with all of the basifiers used in the study. This is not suitable for parenteral administration (without being bound by any theory it is believe that if the pH is too high this would lead to pain on administration). Hence, the pH modulation approach alone does not help in solubilizing QTX125 within the physiological pH range.

### Example 1.2—Cosolvents

In this example the effect of cosolvents on the solubility of QTX125 was examined. The results are given in table 3.

TABLE 3
Formula composition of trials with cosolvents

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T9 | 1% API + 10% Dimethyl | 10 | 8.34 | 0.38 | Turbid pale yellow |
| | acetamide + 30% propylene | | | | suspension |
| | glycol + 59% Phosphate | | | | |
| | buffered saline (pH 7.20) | | | | |
| T10 | 1% API + 10% Dimethyl | 10 | 7.37 | 0.15 | Turbid pale yellow |
| | acetamide + 10% PEG | | | | suspension |
| | 400 + 79% Phosphate | | | | |
| | buffered saline (pH 7.20) | | | | |
| T11 | 1% API + 10% Dimethyl | 10 | 8.43 | 0.36 | Soluble in DMA & |
| | acetamide + 20% | | | | Ethanol mixture, |
| | Ethanol + 69% Phosphate | | | | turns into turbid |
| | buffered saline (pH 7.20) | | | | suspension after |
| | | | | | addition of PBS |
| T12 | 1% API + 30% propylene | 10 | 7.44 | 0.21 | Turbid pale yellow |
| | glycol + 10% PEG | | | | suspension |
| | 400 + 59% Phosphate | | | | |
| | buffered saline (pH 7.20) | | | | |
| T13 | 1% API + 30% propylene | 10 | 8.44 | 0.98 | Turbid pale yellow |
| | glycol + 20% Ethanol + 49% | | | | suspension |
| | Phosphate buffered saline | | | | |
| | (pH 7.20) | | | | |
| T14 | 1% API + 10% PEG | 10 | 7.60 | 0.20 | Turbid pale yellow |
| | 400 + 20% Ethanol + 69% | | | | suspension |
| | Phosphate buffered saline | | | | |
| | (pH 7.20) | | | | |
| T15 | 1% API + 10% Dimethyl | 10 | 8.02 | 0.01 | Soluble in DMA |
| | acetamide + 89% | | | | and turns in to |
| | Phosphate buffered saline | | | | turbid suspension |
| | (pH 7.20) | | | | after PBS addition. |
| T16 | 1% API + 10% Dimethyl | 10 | 6.75 | 0.63 | Pale yellow color |
| | acetamide + 20% | | | | suspension and |
| | Ethanol + 69% of 0.9% | | | | Precipitation |
| | NaCl solution | | | | observed |
| T17 | 1% API + 10% Dimethyl | 10 | 6.94 | 0.32 | Turbid pale yellow |
| | acetamide + 20% | | | | suspension |
| | Ethanol + 69% WFI | | | | |
| T18 | 1% API + 10% Dimethyl | 10 | 5.05 | 2.91 | Precipitation |
| | acetamide + 20% | | | | observed in small |
| | Ethanol + 0.5% PVP | | | | amount |
| | K30 + 68.5% WFI | | | | |
| T19 | 1% API + 30% Propylene | 10 | 7.21 | 2.03 | White to light |
| | glycol + 20% Ethanol + 49% | | | | yellow colored |
| | WFI | | | | turbid suspension |
| T20 | 1% API + 30% Propylene | 10 | 7.07 | 0.75 | White to light |
| | glycol + 20% Ethanol + 49% | | | | yellow colored |
| | of 0.9% NaCl solution | | | | turbid suspension |
| T21 | 10 mg API + 1 mL | 10 | NA | NA | API remains |
| | Soyabean oil | | | | undissolved |
| T22 | 25 mg API + 1.4 mL of N- | 2.5 | 4.86 | 0.26 | Clear solution with |
| | methyl Pyrrolidone + Qs to | | | | dispersed particles |
| | 10 mL with WFI | | | | |

QTX125 remains undissolved or forms turbid suspensions in all the trials executed using different compositions of solvents at their maximum allowable dose. Using a cosolvent approach is not enough to solubilise QTX125.

### Example 1.3—Cyclodextrins

In this example the effect of using formulations comprising cyclodextrin was examined. The results are given in table 4.

TABLE 4
Formula composition of trials with cyclodextrin

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T23 | 1% API + 5% | 10 | 7.16 | 0.14 | White to light yellow |
| | HPβCD + 94% WFI | | | | colored turbid |
| | | | | | suspension |

HPβCD forms turbid suspension and solubility after 24 h is 0.14 mg/mL. The conclusion is that HPβCD alone doesn't help in solubilizing QTX125.

### Example 1.4—Co-Solvents and Micro-Emulsions

In this example the effect of using co-solvents and micro-emulsions was examined. The results are given in table 5.

TABLE 5
Formula composition of trials with cosolvents and Intralipid 20% v/v emulsion

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T24 | 0.5% API + 5% DMA + 10% | 5 | 7.26 | 4.68 | Milky white emulsion |
| | Ethanol + 84.5% Intralipid | | | | |
| | 20% v/v emulsion | | | | |
| T25 | 1% API + 10% DMA + 0.5 mL | 10 | 6.97 | 7.32 | Whitish turbid |
| | of Benzyl alcohol + Intralipid | | | | solution with |
| | 20% v/v emulsion | | | | Undissolved |
| | | | | | particles |
| T26 | 1% API + 20% Ethanol + 79% | 10 | 7.16 | 9.85 | Whitish turbid |
| | Intralipid 20% v/v emulsion | | | | solution with |
| | | | | | Undissolved |
| | | | | | particles |
| T27 | 1% API + 10% DMA + 10% | 10 | 7.14 | 6.07 | Whitish turbid |
| | Ethanol + 79% Intralipid 20% | | | | solution with |
| | v/v emulsion | | | | Undissolved |
| | | | | | particles |
| T28 | 0.5% API + 40% t- | 5 | 6.30 | 4.85 | Pale Yellow colour |
| | Butanol + 59.5% Intralipid | | | | turbid solution |
| | 20% v/v emulsion | | | | |
| T29 | 1% API + 5% DMA + 10% | 10 | 5.39 | 0.22 | Pale white turbid |
| | Ethanol + 2.25% | | | | liquid, particles at |
| | Glycerin + 1.2% Egg | | | | the bottom of vial |
| | lecithin + WFI | | | | |
| T30 | 1% API + 5% DMA + 10% | 10 | 4.67 | 0.05 | Pale white turbid |
| | Ethanol + 2.25% | | | | liquid, particles at |
| | Glycerin + WFI | | | | the bottom of vial |
| T31 | 0.5% API + 5% Dimethyl | 5 | 5.13 | 1.46 | Initially API solution |
| | acetamide + 10% ethanol - | | | | was miscible with |
| | Clear solution after stirring, | | | | Intralipid 20% v/v |
| | Solution added to 0.5 mL of | | | | emulsion. After 15 |
| | Intralipid 20% v/v emulsion | | | | minutes of stirring, |
| | under stirring followed by | | | | emulsion looks like |
| | making the volume up to 1 | | | | broken and oil |
| | mL with Intralipid 20% v/v | | | | droplets seen on the |
| | emulsion | | | | top of emulsion. |
| | | | | | After 24 h - |
| | | | | | Emulsion was |
| | | | | | broken. Oil droplets |
| | | | | | observed. |

It was observed that the pH of the drug solution is within the required range and attained solubility up to 9.85 mg/mL. However, this solution was not stable and it was observed that the emulsion breaks after 24 hours meaning that it is not suitable for the development of a pharmaceutical formulation where it is important that the QTX125 remains in solution during extended periods of storage. To check the reproducibility of T24, trial T31 was executed with the same concentrations of drug and excipients and followed the same manufacturing procedure as that of T24. But it was noticed that results vary from trial to trial and it was not possible to reproducibly obtain the same levels of solubility using this approach. Therefore, it is concluded that this approach is not suitable for development of IV formulation for QTX125.

### Example 1.5—Basifiers and Co-Solvents

In this example the effect of using basifiers and co-solvents was examined. The results are given in table 6.

TABLE 6
Formula composition of trials with basifiers and cosolvents

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T32 | 1% API + 5% Dimethyl | 10 | 10.52 | 8.06 | Pale Yellow |
| | acetamide + 10% | | | | Clear solution |
| | Ethanol + 0.03% WFI + 83.9% | | | | |
| | 1.0N meglumine solution | | | | |
| T33 | 1% API + 5% Dimethyl | 10 | 10.02 | 3.25 | Turbid solution |
| | acetamide + 10% | | | | |
| | Ethanol + 84% 0.1N | | | | |
| | meglumine solution | | | | |
| T34 | 0.5% API + 5% Dimethyl | 5 | 9.71 | 0.40 | Turbid solution |
| | acetamide + 10% | | | | |
| | Ethanol + 0.02% 1.0N | | | | |
| | meglumine solution + 0.02% | | | | |
| | 0.1N HCl + 83.9% WFI | | | | |

### Example 1.6—pH Modulation and Cyclodextrins

In this example the effect of using pH modulation and cyclodextrins was examined. The results are given in table 7.

TABLE 7
Formula composition of trials with pH modulation and cyclodextrin

| | | Strength | | | | |
|---|---|---|---|---|---|---|
| | | of | | | | |
| | | QTX125 | | | | |
| | | added | | | | |
| Trial | Formula | initially | | | Solubility | |
| Number | composition | (mg/mL) | | pH | (mg/mL) | Appearance |
| T35 | 1% API + 20% | 10 | mg | 12.14 | 9.80 | Pale yellow |
| | HPβCD + 300 μl of 1.0N | | | | | color clear |
| | NaOH + volume made up | | | | | solution |
| | with water for injection | | | | | |
| | (WFI) | | | | | |
| T36 | 1% API + 20% | 10 | mg | 11.94 | 10.61 | Pale yellow |
| | HPβCD + 300 μl of 1.0N | | | | | color clear |
| | NaOH + volume made up | | | | | solution |
| | with 0.9% sodium chloride | | | | | |
| | solution | | | | | |
| T37 | 1% API + 20% HPβCD + 10% | 10 | mg | 10.72 | 10.29 | Pale yellow |
| | Meglumine + 69% of WFI | | | | | color clear |
| | | | | | | solution |
| T38 | 1% API + 20% HPβCD + 10% | 10 | mg | 10.71 | 9.63 | Pale yellow |
| | Meglumine + 69% of 0.9% | | | | | color clear |
| | sodium chloride solution | | | | | solution |
| T39 | 1% API + 3.3% | 10 | mg | 11.05 | 8.32 | Clear pale |
| | HPβCD + 95.7% of 1.0N | | | | | yellow solution |
| | meglumine solution (1:1 | | | | | |
| | molar ratio of API to | | | | | |
| | HPβCD) | | | | | |
| T40 | 1% API + 6.6% HPβCD + | 10 | mg | 11.01 | 9.06 | Clear pale |
| | 92.4% of 1.0N meglumine | | | | | yellow solution |
| | solution (1:2 molar ratio of | | | | | |
| | API to HPβCD) | | | | | |
| T41 | 1% API + 3.5% | 10 | mg | 11.27 | 9.31 | Clear pale |
| | SBβCD + 95.5% of 1.0N | | | | | yellow solution |
| | meglumine solution (1:1 | | | | | |
| | molar ratio of API to | | | | | |
| | SBβCD) | | | | | |
| T42 | 1% API + 17.4% | 10 | mg | 11.27 | 9.53 | Clear pale |
| | SBβCD + 81.6% of 1.0N | | | | | yellow solution |
| | meglumine solution (1:5 | | | | | |
| | molar ratio of API to | | | | | |
| | SBβCD) | | | | | |
| T43 | 1% + 17.4% SBβCD + 0.5% | 10 | mg | 11.26 | 8.19 | Clear pale |
| | PVP K30 + 81.1% of 1.0N | | | | | yellow solution |
| | Meglumine solution | | | | | |
| T44 | 1% API + 6.6% HPβCD + 30% | 10 | mg | 10.62 | 8.87 | Turbid |
| | of 1.0N meglumine solution + | | | | | suspension |
| | 62.4% WFI | | | | | |
| T45 | 1% API + 6.6% HPβCD + | 10 | mg | 10.59 | 6.56 | Turbid |
| | 0.5% PVP K30 + 20% of | | | | | suspension |
| | 1.0N meglumine | | | | | |
| | solution + 71.9% WFI | | | | | |
| T46 | 1% API + 17.4% | 10 | mg | 10.79 | 9.75 | Clear pale |
| | SBβCD + 20.0% of 1.0N | | | | | yellow solution |
| | meglumine solution + 61.6% | | | | | |
| | WFI | | | | | |
| T47 | 1% API + 17.4% | 10 | mg | 10.75 | 9.67 | Clear pale |
| | SBβCD + 0.5% PVP | | | | | yellow solution |
| | K30 + 20.0% of 1.0N | | | | | |
| | meglumine solution + 61.1% | | | | | |
| | WFI | | | | | |
| T48 | 1% API + 6.59% | 10 | mg | 10.92 | 4.94 | Pale Yellow |
| | HPβCD + 0.01% WFI + Qs to | | | | | Clear solution |
| | final volume with 1.0N | | | | | |
| | meglumine solution | | | | | |
| T49 | 1% API + 17.38% | 10 | mg | 10.87 | 4.32 | Pale Yellow |
| | SBβCD + 0.02% WFI + 0.04% | | | | | Clear solution |
| | 0.1N acetic Acid + Qs to | | | | | |
| | final volume with 1.0N | | | | | |
| | meglumine solution | | | | | |
| T50 | 0.5% API + 6.59% HPβCD + | 5 | mg | 11.08 | 2.60 | Pale Yellow |
| | 0.01% WFI + Qs to final | | | | | Clear solution |
| | volume with 1.0N | | | | | |
| | Meglumine solution | | | | | |
| T51 | 0.5% API + 0.03% | 5 | mg | 9.99 | 2.38 | Pale Yellow with |
| | WFI + 0.052% Acetic acid, | | | | | dispersed |
| | 0.1N + 17.38% SBβCD + Qs | | | | | particles |
| | to final volume with 1.0N | | | | | |
| | Meglumine solution | | | | | |
| T52 | API + 17.38% SBβCD + | 4 | mg | 9.81 | 2.06 | Pale Yellow with |
| | 0.02% WFI + 0.19% Acetic | | | | | dispersed |
| | acid + Qs to final volume with | | | | | particles |
| | 1.0N Meglumine solution | | | | | |
| | (Strength: 4 mg/mL) | | | | | |
| T53 | Step 1: Combine 0.45 mL | 8 | mg | 9.22 | 3.37 | Solution was |
| | WFI + 8 mg API + 0.05 mL of | | | | | turbid with |
| | 2.0N NaOH + 66 mg HPβCD | | | | | dispersed |
| | Step 2: adjust the pH with | | | | | particles |
| | 0.5 mL of 0.1N HCl | | | | | |
| T54 | Step 1: Combine 0.45 mL | 8 | mg | 8.91 | 2.61 | Slightly turbid |
| | WFI + 8 mg API + 0.05 mL of | | | | | solution |
| | 2.0N NaOH + 66 mg SBβCD | | | | | |
| | Step 2: adjust the pH with | | | | | |
| | 0.5 mL of 0.1N HCl | | | | | |
| T55 | Step 1: Combine 0.45 ml of | 8 | mg | 8.64 | 2.53 | Pink turbid |
| | WFI + 8 mg of API + 0.05 ml | | | | | liquid, particles |
| | of NaOH 2N + 66mg of | | | | | at the bottom of |
| | HPβCD | | | | | vial |
| | Step 2: adjust the pH with | | | | | |
| | 0.56 mL of HCl 0.1N | | | | | |
| T56 | Step 1: Combine 0.45 ml of | 8 | mg | 7.48 | 1.29 | Light Pink turbid |
| | WFI + 8 mg of API + 0.05 ml | | | | | liquid , particles |
| | of NaOH 2N + 66 mg of | | | | | at the bottom of |
| | SBβCD | | | | | vial |
| | Step 2: adjust the pH with | | | | | |
| | 0.57 mL of HCl 0.1N | | | | | |
| T57^{b} | Step 1: Combine 0.45 ml of | 8 | mg | 8.75 | 4.16 | Very slight |
| | WFI + 8 mg of API + 0.05 ml | | | | | turbid solution |
| | of NaOH 2N + 174 mg of | | | | | |
| | SBβCD | | | | | |
| | Step 2: adjust the pH with | | | | | |
| | 0.66 mL of HCl 0.1N - pH | | | | | |
| | dropped to 2.20, Added 3 | | | | | |
| | drops of 0.1N NaOH | | | | | |
| | solution - pH 2.30 - 1.50 | | | | | |
| | mL of this solution was | | | | | |
| | transferred to another vial | | | | | |
| | and analysed for solubility. | | | | | |
| T58 | Trial 55 at pH adjusted to | 8 | mg | 2.30 | 4.75 | Very slight |
| | 2.30 | | | | | turbid solution |
| T59 | Step 1: Combine 0.45 ml of | 4 | mg | 8.14 | 1. 3.75 | Pale yellow |
| | WFI + 4 mg of API + 0.05 ml of | | | | 2. 3.63^{a} | color clear |
| | NaOH 2N + 55.6 mg of | | | | | solution |
| | SBβCD | | | | | |
| | Step 2: adjust the pH with | | | | | |
| | 0.72 mL of HCl 0.1N | | | | | |
| | 1. Unfiltered | | | | | |
| | 2. Filtered through 0.45 μm | | | | | |
| | nylon syringe filter | | | | | |
| T60 | Step 1: Combine 0.2 ml of | 8 | mg | 7.36 | 1. 6.95 | Pale yellow |
| | WFI + 10 mg of API + 0.05 ml | | | | 2. 6.96 | color clear |
| | of NaOH 2N + 200 mg of | | | | | solution |
| | SBβCD | | | | | |
| | Step 2: adjust the pH with | | | | | |
| | 0.72 mL of HCl 0.1N + 0.08 | | | | | |
| | mL of NaOH 0.1N | | | | | |
| | 1. Unfiltered | | | | | |
| | 2. Filtered through 0.45 μm | | | | | |
| | nylon syringe filter | | | | | |
| T61 | Step 1: Combine 0.15 ml of | 10 | mg | 7.61 | 1. 8.91 | Pale yellow |
| | WFI + 10 mg of API + 0.05 ml | | | | 2. 8.85 | color clear |
| | of NaOH 2N + 200 mg of | | | | | solution |
| | SBβCD | | | | | |
| | Step 2: adjust the pH with | | | | | |
| | 0.227 mL of HCl 0.5N + 5 μL | | | | | |
| | of HCl 0.1N + 0.026 mL of | | | | | |
| | NaOH 1.0N | | | | | |
| | 1. Unfiltered | | | | | |
| | 2. Filtered through 0.45 μm | | | | | |
| | nylon syringe filter | | | | | |
| T62 | Step 1: Combine 0.15 ml of | 10 | mg | 7.40 | 1. 9.48 | Pale yellow |
| | WFI + 10 mg of API + 0.06 ml | | | | 2. 9.33 | color clear |
| | of NaOH 1N + 200 mg of | | | | | solution |
| | SBβCD | | | | | |
| | Step 2: adjust the pH with | | | | | |
| | 0.2 mL of HCl 0.25N + 0.02 | | | | | |
| | mL of HCl 0.1N + 5 μL of | | | | | |
| | NaOH 0.1N | | | | | |
| | 1. Unfiltered | | | | | |
| | 2. Filtered through 0.45 μm | | | | | |
| | nylon syringe filter | | | | | |
| T63 | Step 1: Combine 0.20 ml of | 10 | mg | 6.88 | 1. 8.01 | Clear colorless |
| | WFI + 200 mg of SBβCD + 10 | | | | 2. 8.04 | solution |
| | mg of API + 0.06 ml of | | | | | |
| | NaOH 1N | | | | | |
| | Step 2: adjust the pH with | | | | | |
| | 0.21 mL of HCl 0.25N + 0.04 | | | | | |
| | mL of HCl 0.1N | | | | | |
| | 1. Unfiltered | | | | | |
| | 2. Filtered through 0.45 μm | | | | | |
| | nylon syringe filter | | | | | |
| T64 | Step 1: Combine 0.30 ml of | 10 | mg | 7.26 | 9.29 | Pale yellow |
| | WFI + 139 mg of SBβCD + 10 | | | | | color clear |
| | mg of API + 0.05 ml of | | | | | solution |
| | NaOH, 1N | | | | | |
| | Step 2: adjust the pH with | | | | | |
| | 0.19 mL of HCl 0.25N + 0.01 | | | | | |
| | mL of NaOH, 0.1N (1:4 drug | | | | | |
| | to cyclodextrin ratio) | | | | | |
| T65 | Step 1: Combine 0.30 ml of | 10 | mg | 7.45 | 7.54 | Pale yellow |

| | |
|---|---|
| WFI + 104 mg of SBβCD + 10 | color solution, |
| mg of API + 0.06 ml of | precipitation |
| NaOH, 1N | observed after |
| Step 2: adjust the pH with | 24 h holding at |
| 0.26 mL of HCl | room |
| 0.25N + 0.018 mL of HCl, | temperature |
| 0.1N (1:3 drug to | |
| cyclodextrin ratio) | |

^{a}Numbers 1 and 2 in the solubility column of the table refer to the solubility of QTX125 in the solution for the filtered and unfiltered versions.
^{b}In this experiment the pH was dropped to pH 2.2 and then increased to pH 2.3. The solution with a pH of 2.3 was then used to determine the solubility of the compound.

### Example 1.7—pH Modulation and Lyophilization

In this example the effect of using pH modulation and lyophilization was examined. The results are given in table 8.

Samples were prepared as described in the section on the procedure for the solubility trials above and then by lyophilising the solution. The solution was then reconstituted with 10 mL of water and the solubility of QTX125 was determined.

TABLE 8
Formula composition of trials with pH adjustors and Lyophilization

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T66 | 1% API + 15.4% Sodium | 10 | 11.53 | 3.54 | Dark yellow |
| | carbonate + QS to 5 mL | | | | colour clear |
| | with WFI - Lyophilized | | | | solution |
| T67 | 1% API + 15.4% Sodium | 10 | 12.91 | 4.17 | Dark yellow |
| | carbonate + 1.50 mL of | | | | colour clear |
| | 1.0N sodium | | | | solution |
| | hydroxide + QS to 5 mL with | | | | |
| | WFI - Lyophilized | | | | |
| T68 | 1% API + 1 mL of 1.0N | 10 | Part 1. | 6.91 | Clear |
| | sodium hydroxide | | I: 12.67 | | solution |
| | solution + Qs to 10 mL with | | & | | |
| | WFI - Divided into two | | II: 11.82^{a} | | |
| | equal parts, | | Part 2. | 7.57 | Clear |
| | 1. One part kept as | | I: 11.30 | | solution |
| | such and | | & | | |
| | 2. for other part added | | II: 10.49^{a} | | |
| | 0.5 mL of 0.5N | | | | |
| | Acetic acid | | | | |
| | Lyophilized | | | | |

^{a}I refers to the pH measured at time 0; II refers to the pH measured after 24 hours.

### Example 1.8—pH Modulation, Cosolvents and Cyclodextrins

In this example the effect of using pH modulation, Cosolvents and Cyclodextrins was examined. The results are given in table 9.

TABLE 9
Formula composition of trials with pH adjustors, Cosolvents and Cyclodextrins

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T69 | 1% API + 6.6% HPβCD + 5% | 10 mg | 10.88 | 8.79 | Clear pale |
| | dimethyl acetamide + 0.5% | | | | yellow solution |
| | PVP K30 + 10% | | | | |
| | Ethanol + 76.9% of 1.0N | | | | |
| | meglumine solution | | | | |
| T70 | 1% API + 17.4% | 10 mg | 11.21 | 9.27 | Clear pale |
| | SBβCD + 5% dimethyl | | | | yellow solution |
| | acetamide + 0.5% PVP | | | | |
| | K30 + 10% Ethanol + 66.1% | | | | |
| | of 1.0N meglumine solution | | | | |

### Example 1.9—Cosolvents and Cyclodextrins

In this example the effect of using Cosolvents and Cyclodextrins was examined. The results are given in table 10.

TABLE 10
Formula composition of trials with Cosolvents and Cyclodextrins

| | | Strength | | | |
|---|---|---|---|---|---|
| | | of | | | |
| | | QTX125 | | | |
| | | added | | | |
| Trial | Formula | initially | | Solubility | |
| Number | composition | (mg/mL) | pH | (mg/mL) | Appearance |
| T71 | 1% API + 6.6% HPβCD + 5% | 10 mg | 8.51 | 1.00 | Off white turbid |
| | dimethyl acetamide + 10% | | | | suspension |
| | Ethanol + 77.4% of WFI | | | | |
| T72 | 1% API + 17.4% SBβCD + 5% | 10 mg | 4.98 | 0.93 | Off white turbid |
| | dimethyl acetamide + 10% | | | | suspension |
| | Ethanol + 66.6% of WFI | | | | |
| T73 | 1% API + 6.6% HPβCD + 5% | 10 mg | 6.20 | 0.39 | Off white turbid |
| | dimethyl acetamide + 0.5% | | | | suspension |
| | PVP K30 + 10% | | | | |
| | Ethanol + 76.9% of WFI | | | | |
| T74 | 1% API + 17.4% SBβCD + 5% | 10 mg | 5.41 | 0.95 | Off white turbid |
| | dimethyl acetamide + 0.5% | | | | suspension |
| | PVP K30 + 10% | | | | |
| | Ethanol + 66.1% of WFI | | | | |

### Experiment 2—Stability and Lyophilisation Studies

This experiment investigates the development of a robust and stable intravenous injectable formulation for QTX125. This study evaluates the long-term stability of such formulations.

### Materials

The materials used in example 2 are given in table 11.

TABLE 11
Materials used in example 2

| | | Example 2.1 | | Example 2.2 | | Example 2.3 | | Example 2.4 | |
|---|---|---|---|---|---|---|---|---|---|
| | Source | (SF21000162) | | (SF21000182) | | (SF21000273) | | (SF21000175) | |
| | and B. No./ | Qty/ | Qty/ | Qty/ | Qty/ | Qty/ | Qty/ | Qty/ | Qty/ |
| Ingredients | Lot no. | mL | Batch | mL | Batch | mL | Batch | mL | Batch |
| QTX125 | Chemcon | 10 mg | 4.0741 g | 10 mg | 4.0781 g | 10 mg | 2.0373 mg | 10 mg | 4.0752 g |
| | CC-5570.0- | | | | | | | | |
| | 01 | | | | | | | | |
| Betadex | Xi'an DELI | 140 mg | 56.025 g | 278 mg | 112.21 g | 140 mg | 28.012 g | 140 mg | 56.012 g |
| Sulfobutyl | Biochemical | | | | | | | | |
| Ether | Industry | | | | | | | | |
| Sodium | Co., Ltd. | | | | | | | | |
| (SBβCD) | 20200809 | | | | | | | | |
| | (Average | | | | | | | | |
| | molecular | | | | | | | | |
| | weight = | | | | | | | | |
| | 2161.7 | | | | | | | | |
| | g/mol) | | | | | | | | |
| Monosodium | Merck | — | — | — | — | Q.S to | Q.S to | — | — |
| phosphate | DA0D693764 | | | | | pH | pH | | |
| Sodium | Planet | Q.S to | Q.S to | Q.S to | Q.S to | Q.S to | Q.S to | Q.S to | Q.S to |
| hydroxide | Science | pH | pH | pH | pH | pH | pH | pH | pH |
| | RP1912005 | | | | | | | | |
| Hydrochloric | Merck | Q.S to | Q.S to | Q.S to | Q.S to | Q.S to | Q.S to | Q.S to | Q.S to |
| acid | K51440607 | pH | pH | pH | pH | pH | pH | pH | pH |
| Water for | In-house | Q.S to | Q.S to | Q.S to | Q.S to | Q.S to | Q.S to | Q.S to | Q.S to |
| injection | | mL | 400 mL | mL | 400 mL | mL | 200 mL | mL | 400 mL |

### HPLC Measurement Method

The parameters required for the HPLC method used to determine the concentration of QTX125 in solution is set out below.

| Chemical/Reagent Name | Grade | Supplier |
|---|---|---|
| Orthophosphoric Acid | AR Grade | Merck |
| DMSO | HPLC Grade | Spectrochem |
| Methanol | HPLC grade | Merck |
| Acetonitrile | HPLC grade | Merck |
| Water (HPLC grade or Milli Q | HPLC (Milli Q) | Evoqua |
| water) | | |

### Instrumentation and Equipment:

| System | Waters HPLC Alliance |
|---|---|
| Pump | Quaternary pump/Binary pump |
| Detector | UV/PDA detector |

### Chromatographic Parameters:

| HPLC Column | Accucore C18 150 mm × 4.6 mm; 2.6 μm | | |
|---|---|---|---|
| | (or) Equivalent column | | |
| | (Make: Thermo) | | |
| Column Temperature | 25 ± 2° C. | | |
| Sampler temperature | 20 ± 2° C. | | |
| Detector Wavelength | 254 nm | | |
| Flow rate | 1.0 ml/min | | |
| Injection Volume | 5 μL | | |
| Run time | 55 minutes | | |
| Mobile Phase | Mobile phase A: 0.1% OPA in water | | |
| | Mobile phase B: Acetonitrile:Methanol | | |
| | (80:20% v/v) | | |
| | Time | | |
| | (minutes) | % A | % B |
| Gradient Condition | 0.0 | 95.0 | 5.0 |
| | 2.0 | 95.0 | 5.0 |
| | 37.0 | 50.0 | 50.0 |
| | 39.0 | 20.0 | 80.0 |
| | 47.0 | 20.0 | 80.0 |
| | 48.0 | 95.0 | 5.0 |
| | 55.0 | 95.0 | 5.0 |

| | |
|---|---|
| Diluent | 0.1% OPA:Methanol (50:50 v/v) |

HPLC determination of the percentage of QTX125 in the solutions below was determined using the protocols set out above. Peaks for QTX125 were seen at a retention time of approximately 24.1 min.

Assay (% LC) was determined by comparing the area of the peak for QTX125 to a calibration curve determined by running samples of known concentration of QTX125 (standards) on the HPLC.

QTX125% was calculated based on the area of the QTX125 peak compared to the sum of all peak areas in the chromatogram.

Percentage impurities (% w/w) were calculated based on the area normalization method. This is based on the relative area of the impurity peaks (i.e. peaks which elute with a retention time not at 24.1 minutes) compared to the sum of all peak areas in the chromatogram.

### Example 2.1—1:2.7 Liquid Injection Formulation

### Manufacturing Process

The liquid injection formulations used in example 2.1 were manufactured as follows for a batch size of 400 mL:
30% v/v of water based on the total volume of the formulation was added to a beaker;
The required quantity of SBβCD was added to the beaker and dissolved by stirring for 20 minutes. The pH of the solution was checked.
20 mL of 1.0 N NaOH (corresponding to 5% v/v NaOH) was added to the solution (800 mg of sodium hydroxide) and mixed by stirring for 15 minutes. The pH of the solution was checked.
The appropriate amount of QTX125 was added into the solution and dissolved by stirring for 40 minutes at room temperature. The pH of the solution was checked and adjusted to pH 7.3 using 0.25N HCl solution.
The volume of bulk solution was made up to 100% v/v of the total batch size with water for injection and stirred for 15 minutes. The pH of the solution was checked.
The solution was filtered through a 0.2 μm PVDF filter membrane (Merck).

The stability results for the liquid formulation with a 1:2.7 ratio of drug to SBβCD after 1 month (1 M), 2 months (2 M) and 3 months (3 M) of storage at 5±3° C., 25° C. and 60% relative humidity (25° C./60% RH) and 40° C. and 75% relative humidity (40° C./75% RH) are given in table 12.

TABLE 12
Stability results of the 1:2.7 liquid formulation

| | Condition | | | | | | | | | |
|---|---|---|---|---|---|---|---|---|---|---|
| Test | | 5 ± 3° C. | | | 25° C./60% RH | | | 40° C./75% RH | | |
| parameters | Initial | 1 M | 2 M | 3 M | 1 M | 2 M | 3 M | 1 M | 2 M | 3 M |
| Description | Clear pale | Clear pale straw | | | Cloudy straw | | | Dark cloudy straw | | |
| | straw | colored solution | | | colored solution with | | | colored solution with | | |
| | colored | | | | dispersed particles | | | dispersed particles | | |
| | solution | | | | | | | | | |
| pH | 7.29 | 7.26 | 7.27 | 7.18 | 6.21 | 4.56 | 4.47 | 6.98 | 6.69 | 6.31 |
| Assay | 98.6 | 99.3 | 100.2 | 97.2 | 97.4 | 98.5 | 97.3 | 97.6 | 97.8 | 95 |
| (% LC) | | | | | | | | | | |

Related Substances (%)

| | | | | | | | | | | |
|---|---|---|---|---|---|---|---|---|---|---|
| QTX125 | 99.21 | 99.15 | 99.035 | 99.065 | 98.897 | 98.938 | 98.799 | 98.186 | 97.318 | 96.707 |
| Total | 0.793 | 0.8 | 0.828 | 0.796 | 1.026 | 0.963 | 1.132 | 1.744 | 2.503 | 3.194 |
| impurities | | | | | | | | | | |

Precipitation was observed in samples stored at 25° C./60% RH and 40° C./75% RH. A decrease in pH was observed in the 25° C./60% RH and 40° C./75% RH samples. Though the formulation appears to be stable for up to 3 months at 2-8° C., it was found to be unstable at 25° C./60% RH and 40° C./75% RH which is evident from the description of the solution, drop in pH and increase in total impurities.

### Example 2.2—1:5.4 Liquid Injection Formulation

Samples were manufactured as described above for example 2.1. The stability results for the liquid formulation with a 1:5.4 ratio of drug to SBβCD after 1 month (1 M), 2 months (2 M) and 3 months (3 M) of storage at 5±3° C., 25° C. and 60% relative humidity (25° C./60% RH) and 40° C. and 75% relative humidity (40° C./75% RH) are given in table 13.

TABLE 13
Stability results of the 1:5.4 liquid injection formulation

| | Condition | | | | | | | | | |
|---|---|---|---|---|---|---|---|---|---|---|
| Test | | 5 ± 3° C. | | | 25° C./60% RH | | | 40° C./75% RH | | |
| parameters | Initial | 1 M | 2 M | 3 M | 1 M | 2 M | 3 M | 1 M | 2 M | 3 M |
| Description | Clear | Clear pale straw | | | Clear pale straw | | | Clear Dark straw | | |
| | pale | colored solution | | | colored solution | | | colored solution | | |
| | straw | | | | | | | | | |
| | colored | | | | | | | | | |
| | solution | | | | | | | | | |
| pH | 7.39 | 7.2 | 7.24 | 7.22 | 7.15 | 7.13 | 7.04 | 6.96 | 6.82 | 6.94 |
| Assay | 98.4 | 98.6 | 98 | 96.6 | 98.4 | 97.5 | 95.7 | 94.1 | 93.9 | 91.1 |
| (% LC) | | | | | | | | | | |

Related Substances (%)

| | | | | | | | | | | |
|---|---|---|---|---|---|---|---|---|---|---|
| QTX125 | 98.973 | 98.927 | 98.886 | 98.936 | 98.569 | 98.312 | 99.065 | 97.345 | 96..098 | 95.592 |
| Total | 0.94 | 1.036 | 1.005 | 0.866 | 1.319 | 1.65 | 0.853 | 2.55 | 3.841 | 4.233 |
| impurities | | | | | | | | | | |

### Example 2.3—1:2.7 Liquid Injection with Buffer

### Manufacturing Process

The liquid injection formulations used in example 2.3 were manufactured as follows for a batch size of 400 mL:
30% v/v of water based on the total volume of the formulation was added to a beaker;
The required quantity of SBβCD was added to the beaker and dissolved by stirring for 20 minutes. The pH of the solution was checked.
10 mL of 1.0 N NaOH (2.5% v/v NaOH) was added to the solution (400 mg of sodium hydroxide) and mixed by stirring for 15 minutes. The pH of the solution was checked.
The appropriate amount of QTX125 was added into the solution and dissolved by stirring for 40 minutes at room temperature. The pH of the solution was checked and adjusted to pH 7.3 using 0.25N HCl solution.
120 mL of 0.2 N monosodium phosphate solution (30% v/v) was added to the mixture and mixed for 15 minutes. The pH of the solution was checked.
The volume of bulk solution was made up to 100% v/v of the total batch size with water for injection and stirred for 15 minutes. The pH of the solution was checked.
The solution was filtered through a 0.2 μm PVDF filter membrane (Merck).

The stability results for the liquid formulation with a 1:2.7 ratio of drug to SBβCD in buffer after 1 month (1 M), 2 months (2 M) and 3 months (3 M) of storage at 5±3° C., 25° C. and 60% relative humidity (25° C./60% RH) and 40° C. and 75% relative humidity (40° C./75% RH) are given in table 14.

TABLE 14
Stability results of the 1:2.7 liquid injection with buffer

| | Condition | | | | | | | | | |
|---|---|---|---|---|---|---|---|---|---|---|
| | | 5 ± 3° C. | | | 25° C./60% RH | | | 40° C./75% RH | | |
| | Initial | 1 M | 2 M | 3 M | 1 M | 2 M | 3 M | 1 M | 2 M | 3 M |
| Description | Pale | Pale | Dark | Clear pale | Pale | Dark | Pale | Light | Dark | Brownish |
| | Straw | Straw | brown | yellow | brown | brown | yellow | brown | brown | colored |
| | colored | colored | colored | colored | colored | colored | colored | colored | colored | cloudy and |
| | solution | solution | solution | solution | solution | solution | solution | solution | solution | turbid |
| | with | with | with | with solid | with | with | with solid | with | with | solution |
| | Lumps | Lumps | Lumps | lumps | Lumps | Lumps | particles | Lumps | Lumps | with solid |
| | | | | | | | | | | particles |
| Reconstituted | 7.6 | 7.62 | Not | Not | 7.57 | Not | Not | 7.51 | Not | Not |
| solution pH | | | analyzed | analyzed | | analyzed | analyzed | | analyzed | analyzed |
| Assay (% LC) | 96.4 | 97.2 | | | 98.8 | | | 82.9 | | |

Related Substances (%)

| | | | | | | | | | | |
|---|---|---|---|---|---|---|---|---|---|---|
| QTX125 | 99.28 | 99.016 | Not | Not | 98.421 | Not | Not | 97.362 | Not | Not |
| Total | 0.692 | 0.797 | analyzed | analyzed | 1.258 | analyzed | analyzed | 2.424 | analyzed | analyzed |
| impurities | | | | | | | | | | |

Lumps were seen in the samples after the second and third months; therefore, these samples were not tested.

### Example 2.4—1:2.7 Lyophilised Product

Samples were manufactured as described above for example 2.1 and then filled into 20 mL/20 mm amber USP type I vials, half stoppered using 20 mm rubber stoppers and loaded into a lyophilizer.

The lyophilization process was carried out using the following program, as set out in table 15.

TABLE 15
Lyophilization process used to prepare
the samples for stability testing

| | | Temperature | Time | Vacuum | Ramp/ |
|---|---|---|---|---|---|
| Stages | Steps | (° C.) | (Mins) | (mTorr) | Hold |
| Thermal | 1 | 25 | 10 | — | H |
| treatment | 2 | −5 | 120 | — | R |
| | 3 | −5 | 60 | — | H |
| | 4 | −40 | 140 | — | R |
| | 5 | −40 | 360 | — | H |
| Extra | Condenser | −40 | 20 | 200 | — |
| freeze | temperature: | | | | |
| | −50° C. | | | | |
| Drying | 1 | −40 | 10 | 200 | H |
| | 2 | −15 | 300 | 100 | R |
| | 3 | −15 | 900 | 100 | H |
| | 4 | −5 | 180 | 100 | R |
| | 5 | −5 | 240 | 100 | H |
| | 6 | 15 | 180 | 100 | R |
| | 7 | 15 | 360 | 100 | H |
| | 8 | 30 | 240 | 100 | R |
| | 9 | 30 | 600 | 100 | H |
| Post heat | 10 | 35 | 800 | 100 | — |

The stability results for the reconstituted lyophilised formulation with a 1:4 ratio of drug to SBβCD after 1 month (1 M), 2 months (2 M) and 3 months (3 M) of storage at 5±3° C., 25° C. and 60% relative humidity (25° C./60% RH) and 40° C. and 75% relative humidity (40° C./75% RH) are given in table 16.

TABLE 16
Stability results of the 1:2.7 lyophilised formulation

| | Conditions | | | | | | | | | |
|---|---|---|---|---|---|---|---|---|---|---|
| Test | | 5 ± 3° C. | | | 25° C./60% RH | | | 40° C./75% RH | | |
| parameters | Initial | 1 M | 2 M | 3 M | 1 M | 2 M | 3 M | 1 M | 2 M | 3 M |
| Description | Off white colored lyophilized cake | | | | | | | | | |
| of lyophilized | | | | | | | | | | |
| cake | | | | | | | | | | |
| Reconstitution | 48 sec | 42 sec | ND | 46 sec | 50 sec | ND | 51 sec | 46 sec | ND | 45 sec |
| time | | | | | | | | | | |
| Reconstituted | Clear | Clear | ND | Clear | Clear | ND | Clear | Clear | ND | Clear |
| solution | pale | pale | | pale | pale | | pale | pale | | pale |
| Description | straw | straw | | straw | straw | | straw | straw | | straw |
| | colored | colored | | colored | colored | | colored | colored | | colored |
| | solution | solution | | solution | solution | | solution | solution | | solution |
| Reconstituted | 7.41 | 7.44 | ND | 7.51 | 7.45 | ND | 7.54 | 7.46 | ND | 7.52 |
| solution pH | | | | | | | | | | |
| Water | 1.17 | 1.61 | ND | 1.77 | 1.27 | ND | 1.65 | 1.26 | ND | 1.68 |
| content | | | | | | | | | | |
| Assay (% LC)^{a} | 101.4 | 100.1 | 105.5 | 101.8 | 101.5 | 106.2 | 100.2 | 100.2 | 103.1 | 97.5 |

Related Substances (%)

| | | | | | | | | | | |
|---|---|---|---|---|---|---|---|---|---|---|
| QTX125 | 99.19 | 99.21 | 98.97 | 99.32 | 99.15 | 98.91 | 99.398 | 99.17 | 98.85 | 99.358 |
| Total | 0.808 | 0.793 | 0.909 | 0.606 | 0.852 | 0.977 | 0.528 | 0.832 | 0.997 | 0.563 |
| impurities | | | | | | | | | | |

^{a}The assay was determined by comparison to a standard of known weight of QTX125

No changes were observed in the tested parameters. The drug product in the lyophilised cake form is found to be stable up to 3 months under all three conditions (2° C.-8° C., 25° C./60% RH and 40° C./75% RH).

### Conclusions of Example 2

In conclusion, the lyophilized drug product is comparatively stable over the ready to use injection form. A summary of the observations from each of the examples 2.1-2.4 is given in table 17 below.

TABLE 17
Summary of observations

| | Drug to | Observations | | |
|---|---|---|---|---|
| | SBβCD | | 25° C./ | 40° C./ |
| Example | ratio | 5 ± 3° C. | 60% RH | 75% RH |
| 2.1 | 1:2.7 | Stable up | Unstable | Unstable |
| (SF21000162) | (liquid | to 3 M | | |
| | injection) | | | |
| 2.4 | 1:2.7 | Stable up | Stable up | Stable up |
| (SF21000175) | (Lyo product) | to 3 M | to 3 M | to 3 M |
| 2.2 | 1:5.4 | Stable up | Stable up | Unstable |
| (SF21000182) | (liquid | to 3 M | to 3 M | |
| | injection) | | | |
| 2.3 | 1:2.7 | Unstable - | Unstable - | Unstable - |
| (SF21000273) | (liquid | precipi- | precipi- | precipi- |
| | injection | tation | tation | tation |
| | with buffer) | observed | observed | observed |

### Example 3—Tolerance of New Formulation in Sprague Dawley Rats

The object of this study was to evaluate the tolerance of mammals (male Sprague Dawley rats) to various formulations of QTX125.

### Formulations Used in this Example

Formula A: 5% v/v 1 N NaOH+13.9% SBβCD (Captisol)+adjust the pH with 0.25 N HCl and/or 0.1 NaOH to 7.2+water for Injection q.s
Formula B: 20% PEG 400+30% propylene glycol+15% glycerol+10% Tween 80+25% water for injection q.s

The study design for this study is summarised in table 18 below.

TABLE 18
Study design for testing the tolerance of rats to various
dosage forms of the different QTX125 formulations.

| | | | Ratio of | | |
|---|---|---|---|---|---|
| | No. | Nominal | cyclodextrin | Dose | |
| | of | Dose | (SBβCD) to API | Volume | Strength |
| Treatment | Rats | (mg/kg) | (Mw 2162 g/mol) | (mL/kg) | (mg/mL) |
| Formula A | 3 | 15 | 1:35 | 20 | 0.750 |
| | 3 | 30 | 1:18 | | 1.5 |
| | 3 | 40 | 1:13 | | 2.0 |
| | 3 | 60 | 1:8.9 | | 3.0 |
| | 3 | 80 | 1:6.6 | | 4.0 |
| | 3 | 120 | 1:4.4 | | 6.0 |
| | 3 | 200 | 1:2.7 | | 10.0 |
| Formula B | 3 | 30 | — | | 1.5 |

Formulations were prepared on the day of dosing. The formulations were administered to the respective group of animals as intravenous infusions for 30 minutes using a Harvard pump infusion through a femoral vein cannula.

### Results

Intravenous infusion of formula A (Doses 15, 30, 40, 60, 80, 120 and 200 mg/kg) was well tolerated by the male Sprague Dawley rats.

Following single intravenous infusion of formula B to male Sprague Dawley rats (Dose: 30 mg/kg) all animals were found dead during the infusion period.

These results demonstrate that formula A is better tolerated in male Sprague Dawley rats than formula B.

1. An aqueous pharmaceutical formulation for injection comprising:
a compound of formula I, 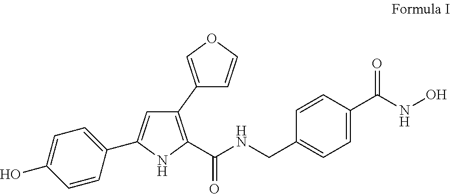 [SMILES: O=C(NO)c1ccc(CNC(=O)c2[nH]c(-c3ccc(O)cc3)cc2-c2ccoc2)cc1]
and a compound of formula II, 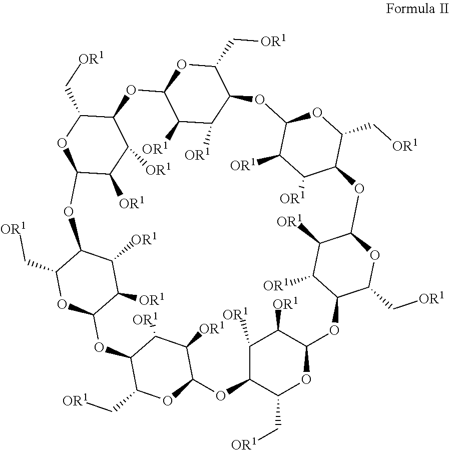
wherein each R¹ is independently selected from the group of: —H or 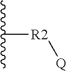
wherein R² is either absent or is a C₁₋₄alkyl;
Q is selected from the group of: —H, —SO₃⁻, —OH, —C(O)R³ or —C(OH)R³₂; and
R³ is independently selected from —H or C₁₋₄alkyl;
wherein the molar ratio of the compound of formula I to the compound of formula II is from 1:50-1:2; and
wherein the pH of the pharmaceutical formulation is between pH 7 and pH 8.

2. The aqueous pharmaceutical formulation of claim 1, wherein the compound of formula I is dissolved at a concentration of 7.5 mg/mL or more.

3. The aqueous formulation of claim 1, wherein the compound of formula I is dissolved at a concentration of 8 mg/mL or more.

4. The aqueous pharmaceutical formulation of claim 1, wherein the compound of formula II is selected from the group of: β-cyclodextrin, (C₁₋₄alkyl)-β-cyclodextrin, (hydroxy-C₁₋₄alkyl)-β-cyclodextrin and sulfobutyl ethers of β-cyclodextrin.

5. The aqueous pharmaceutical formulation of claim 1, wherein the compound of formula II is hydroxy propyl β cyclodextrin or sulfobutyl ether β cyclodextrin (SBβCD).

6. The aqueous pharmaceutical formulation of claim 1, wherein the compound of formula II is sulfobutyl ether β-cyclodextrin (SBβCD).

7. The aqueous pharmaceutical formulation of claim 1, wherein the molar ratio of the compound of formula I to the compound of formula II is from 1:40 to 1:2.5.

8. The aqueous pharmaceutical formulation of claim 7, wherein the molar ratio of the compound of formula I to the compound of formula II is from 1:15 to 1:2.5.

9. The aqueous pharmaceutical formulation of claim 1, wherein:
the compound of formula I is dissolved at a concentration of 9 mg/mL or more;
the compound of formula II is sulfobutyl ether β-cyclodextrin; and
the molar ratio of the compound of formula I to sulfobutyl ether β-cyclodextrin is from 1:4.5 to 1:2.5.

10. The aqueous pharmaceutical formulation for injection of claim 1 comprising:
a compound of formula I, 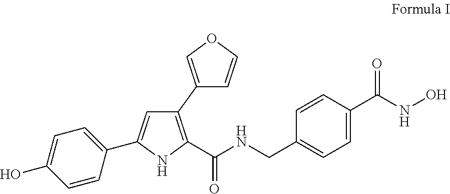 [SMILES: O=C(NO)c1ccc(CNC(=O)c2[nH]c(-c3ccc(O)cc3)cc2-c2ccoc2)cc1]
and compound of formula II, 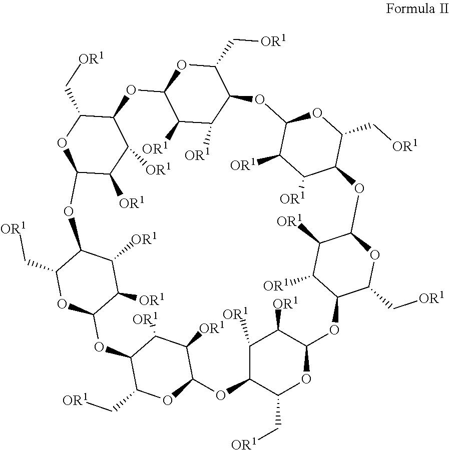
wherein each R¹ is independently selected from the group of: —H or 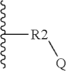
wherein R² is absent or is a C₁₋₄alkyl;
Q is selected from the group of: —H, —SO₃⁻, —OH, —C(O)R³ or —C(OH)R³₂ and
R³ is independently selected from —H or is a C₁₋₄alkyl
wherein the pharmaceutical formulation is obtainable by a method including:
Step 1: preparing a mixture comprising water, a compound of formula I and a compound of formula II, wherein the molar ratio of the compound of formula I to the compound of formula II is from 1:50-1:2;
Step 2: adding an acid to lower the pH of the mixture to a value between pH 7 and pH 8;
optionally wherein step 1 is carried out at a pH of 9 or more.

11. A dry pharmaceutical formulation obtainable by drying the aqueous pharmaceutical formulation of claim 1.

12. The dry pharmaceutical formulation of claim 11, obtainable by lyophilising the aqueous pharmaceutical formulation.

13. A kit comprising the dry pharmaceutical formulation of claim 11 and a pharmaceutically acceptable grade of water, buffer solution or saline solution for use in reconstituting the aqueous pharmaceutical formulation.

14. (canceled)

15. (canceled)

16. (canceled)

17. (canceled)

18. A method of treatment comprising administering an aqueous pharmaceutical formulation of claim 1 to a patient in need of such treatment.

19. The method of treatment of claim 18, wherein the method is for the treatment of cancer or an autoimmune disease.

20. A method of manufacture of an aqueous pharmaceutical formulation of claim 1, comprising:
Step 1: preparing a mixture comprising water, a compound of formula I and a compound of formula II;
Step 2: adding an acid to lower the pH of the mixture to a value between pH 7 and pH 8.

21. The method of claim 20, wherein step 1 is carried out at a pH of 9 or above.

22. The method of claim 20, further comprising a step of lyophilising the aqueous pharmaceutical formulation obtainable after step 2.

23. The method of claim 20, wherein the method further comprises a step of storing the aqueous pharmaceutical formulation for a period of at least three months at room temperature.
